(19) United States
(12) Patent Application Publication
(10) Pub. No.: US 2002/0146439 A1
(43) Pub. Date: Oct. 10, 2002

(54) Compositions and methods for treating hair loss using oximyl and hydroxylamino prostaglandins

(76) Inventors: Mitchell Anthony DeLong, West Chester, OH (US); John McMillan McIver, Cincinnati, OH (US); Robert Scott Youngquist, Mason, OH (US)

Correspondence Address: Catherine U. Brown, The Proctor & Gamble Company, Miami Valley Laboratories, P.O. Box 538707, Cincinnati, OH, 45253-8707, US

(21) Appl. No.: 09/774,556

(22) Filed: Jan. 31, 2001

Related U.S. Application Data
(60) Provisional application No. 60/193,844, filed on Mar. 31, 2000.

(51) Int. Cl. A61K 7/06; A61K 7/00

(52) U.S. Cl. 424/401; 424/70.1

(57) ABSTRACT

A method for treating hair loss in mammals uses compositions containing prostaglandins analogs. The compositions can be applied topically to the skin. The compositions can arrest hair loss, reverse hair loss, and promote hair growth.

### FIELD OF THE INVENTION

This invention relates to compositions and methods for treating hair loss in mammals. More particularly, this invention relates to compositions and methods for arresting or reversing hair loss, or both, and promoting hair growth.

### BACKGROUND OF THE INVENTION

Hair loss is a common problem which is, for example, naturally occurring or chemically promoted through the use of certain therapeutic drugs designed to alleviate conditions such as cancer. Often such hair loss is accompanied by lack of hair re-growth which causes partial or full baldness.

Hair growth on the scalp does not occur continuously, but rather occurs by a cycle of activity involving alternating periods of growth and rest. This cycle is divided into three main stages; anagen, catagen, and telogen. Anagen is the growth phase of the cycle and is characterized by penetration of the hair follicle deep into the dermis with rapid proliferation of cells which are differentiating to form hair. The next phase is catagen, which is a transitional stage marked by the cessation of cell division, and during which the hair follicle regresses through the dermis and hair growth ceases. The next phase, telogen, is characterized as the resting stage during which the regressed follicle contains a germ with tightly packed dermal papilla cells. At telogen, the initiation of a new anagen phase is caused by rapid cell proliferation in the germ, expansion of the dermal papilla, and elaboration of basement membrane components. When hair growth ceases, most of the hair follicles reside in telogen and anagen is not engaged, thus causing the onset of full or partial baldness.

Attempts to invoke the re-growth of hair have been made by, for example, the promotion or prolongation of anagen. Currently, there are two drugs approved by the United States Food and Drug Administration for the treatment of male pattern baldness: topical minoxidil (marketed as ROGAINE® by Pharmacia & Upjohn), and oral finasteride (marketed as PROPECIA® by Merck & Co., Inc.). However, the search for efficacious hair growth inducers is ongoing due to factors including safety concerns and limited efficacy.

The thyroid hormone thyroxine (“T4”) converts to thyronine (“T3”) in human skin by deiodinase I, a selenoprotein. Selenium deficiency causes a decrease in T3 levels due to a decrease in deiodinase I activity; this reduction in T3 levels is strongly associated with hair loss. Consistent with this observation, hair growth is a reported side effect of administration of T4. See, e.g., Berman, “Peripheral Effects of L-Thyroxine on Hair Growth and Coloration in Cattle”, Journal of Endocrinology, Vol. 20, pp. 282-292 (1960); and Gunaratnam, “The Effects of Thyroxine on Hair Growth in the Dog”, J. Small Anim. Pract., Vol. 27, pp. 17-29 (1986). Furthermore, T3 and T4 have been the subject of several patent publications relating to treatment of hair loss. See, e.g., Fischer et al., DE 1,617,477, published Jan. 8, 1970; Mortimer, G B 2,138,286, published Oct. 24, 1984; and Lindenbaum, WO 96/25943, assigned to Life Medical Sciences, Inc., published Aug. 29, 1996.

Unfortunately, however, administration of T3 or T4, or both, to treat hair loss is often not practicable because these thyroid hormones can induce significant cardiotoxicity. See, e.g., Walker et al., U.S. Pat. No. 5,284,971, assigned to Syntex, issued Feb. 8, 1994 and Emmett et al., U.S. Pat. No. 5,061,798, assigned to Smith Kline & French Laboratories, issued Oct. 29, 1991.

In an alternative approach, prostaglandins have been proposed to promote hair growth because prostaglandins may have a similar benefit to thyroid hormones, i.e., increasing hair length and changing pigmentation. Naturally occurring prostaglandins (e.g., PGA₂, PGB₂, PGE₁, PGF_{2α}, and PGI₂) are C-20 unsaturated fatty acids. PGF_{2α}, the naturally occurring Prostaglandin F analog in humans, is characterized by hydroxyl groups at the C9 and C11 positions on the alicyclic ring, a cis-double bond between C5 and C6, and a trans-double bond between C13 and C14. PGF_{2α} has the formula:

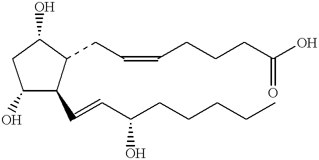

Analogs of naturally occurring Prostaglandin F are known in the art. For example, see U.S. Pat. No. 4,024,179 issued to Bindra and Johnson on May 17, 1977; German Patent No. DT-002,460,990 issued to Beck, Lerch, Seeger, and Teufel published on Jul. 1, 1976; U.S. Pat. No. 4,128,720 issued to Hayashi, Kori, and Miyake on Dec. 5, 1978; U.S. Pat. No. 4,011,262 issued to Hess, Johnson, Bindra, and Schaaf on Mar. 8, 1977; U.S. Pat. No. 3,776,938 issued to Bergstrom and Sjovall on Dec. 4, 1973; P. W. Collins and S. W. Djuric, “Synthesis of Therapeutically Useful Prostaglandin and Prostacyclin Analogs”, Chem. Rev. Vol. 93 (1993), pp. 1533-1564; G. L. Bundy and F. H. Lincoln, “Synthesis of 17-Phenyl-18,19,20-Trinorprostaglandins: I. The PG₁ Series”, Prostaglandin, Vol. 9 No. 1 (1975), pp. 1-4; W. Bartman, G. Beck, U. Lerch, H. Teufel, and B. Scholkens, “Luteolytic Prostaglandin: Synthesis and Biological Activity”, Prostaglandin, Vol. 17 No. 2 (1979), pp. 301-311; C. liljebris, G. Selen, B. Resul, J. Sternschantz, and U. Hacksell, “Derivatives of 17-Phenyl-18, 19,20-trinorprostaglandin F₂α. Isopropyl Ester: Potential Antiglaucoma Agents”, Journal of Medicinal Chemistry, Vol. 38 No. 2 (1995), pp. 289-304.

Prostaglandins in general have a wide range of biological activities. For example, PGE₂ has the following properties: a) regulator of cell proliferation, b) regulator of cytokine synthesis, c) regulator of immune responses and d) inducer of vasodilatation. Vasodilatation is thought to be one of the mechanisms of how minoxidil provides a hair growth benefit. In vitro results in the literature also indicate some anti-inflammatory properties of the prostaglandins; c.f., Tanaka, H. Br J. Pharm. (1995) 116, 2298.

However, previous attempts at using prostaglandins to promote hair growth have been unsuccessful. Different prostaglandin analogs can bind to multiple receptors at various concentrations with a biphasic effect. Furthermore, administration of naturally occurring prostaglandins can cause side effects such as inflammation, surface irritation, smooth muscle contraction, pain, and bronchoconstriction. Therefore, it is an object of this invention to provide methods for using prostaglandin analogs to grow hair and to provide compositions that promote hair growth in humans and lower animals. It is a further object of this invention to provide a selection of appropriate prostaglandin analogs that will promote hair growth and that do not cause significant undesirable. side effects.

### SUMMARY OF THE INVENTION

This invention relates to compositions and methods for treating hair loss. The methods comprise administering a composition comprising a specific prostaglandin that interacts strongly with hair-selective receptors, such as the FP receptor. The choice of prostaglandin is important because the prostaglandin must selectively activate the FP receptor and not activate any other receptors that would negate the effect of activating the FP receptor. The compositions comprise: component A) the prostaglandin, component B) a carrier, and optionally component C) an activity enhancer.

Suitable prostaglandins are selected from the group consisting of oximyl-prostaglandins and hydroxylamino-prostaglandins. These oximyl- and hydroxylamino-prostaglandins have the general formula:

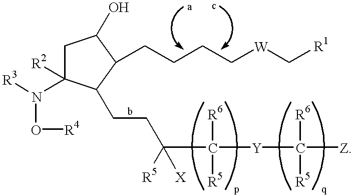

Preferably, W is an oxygen atom or an alkyl group; X is OR⁸; Y is a bond or an oxygen atom; Z is thienyl or phenyl; R¹ is CO₂H, CO₂R⁷, or C(O)NHOH; R² and R³ form a covalent bond; R⁴ is a lower monovalent hydrocarbon group; R⁵ H or CH₃; R⁶ is H or CH₃; R⁷ is a methyl, ethyl, or isopropyl group; and R⁵ is preferably a hydrogen atom. Preferably, p is an integer with a value of 1 to 5, and q is an integer with a value of 0 to 5. Preferably, bond a is a single bond, a cis double bond, or a trans double bond, bond b is a single bond or a trans double bond, and bond c is a cis double bond or a trans double bond.

### DETAILED DESCRIPTION OF THE INVENTION

This invention relates to compositions and methods using oximyl- and hydroxylamino-prostaglandins to treat hair loss in mammals. “Treating hair loss” includes arresting hair loss or reversing hair loss, or both, and promoting hair growth.

Publications and patents are referred to throughout this disclosure. All U.S. Patents cited herein are hereby incorporated by reference.

All percentages, ratios, and proportions used herein are by weight unless otherwise specified.

### Definition and Usage of Terms

The following is a list of definitions for terms, as used herein:

“Activate” means binding and signal transduction of a receptor.

“Acyl group” means a monovalent group suitable for acylating a nitrogen atom to form an amide or carbamate, an alcohol to form a carbonate, or an oxygen atom to form an ester group. Preferred acyl groups include benzoyl, acetyl, tert-butyl acetyl, para-phenyl benzoyl, and trifluoroacetyl. More preferred acyl groups include acetyl and benzoyl. The most preferred acyl group is acetyl.

“Alkoxy group” means a monovalent group having the structure —O(CₓH₂ₓ₊₁) wherein x is 1 to 12.

“Aromatic group” means a monovalent group having a monocyclic ring structure or fused bicyclic ring structure. Monocyclic aromatic groups contain 5 to 10 carbon atoms, preferably 5 to 7 carbon atoms, and more preferably 5 to 6 carbon atoms in the ring. Bicyclic aromatic groups contain 7 to 17 carbon atoms, preferably 7 to 14 carbon atoms, and more preferably 9 or 10 carbon atoms in the ring. Aromatic groups are unsubstituted. The most preferred aromatic group is phenyl.

“Carbocyclic group” means a monovalent saturated or unsaturated hydrocarbon ring. Carbocyclic groups are monocyclic, or are fused, spiro, or bridged bicyclic ring systems. Monocyclic carbocyclic groups contain 4 to 10 carbon atoms, preferably 4 to 7 carbon atoms, and more preferably 5 to 6 carbon atoms in the ring. Bicyclic carbocyclic groups contain 7 to 17 carbon atoms, preferably 7 to 14 carbon atoms, and more preferably 9 to 10 carbon atoms in the ring. Carbocyclic groups are unsubstituted. Preferred carbocyclic groups include cyclopentyl, cyclohexyl, cyclohexenyl, cycloheptyl, and cyclooctyl. More preferred carbocyclic groups include cyclohexyl, cycloheptyl, and cyclooctyl. The most preferred carbocyclic group is cycloheptyl. Carbocyclic groups are not aromatic.

“Cyano group” means a group containing a nitrile functionality.

“FP agonist” means a compound that activates the FP receptor.

“FP receptor” means known human FP receptors, their splice variants, and undescribed receptors that have similar binding and activation profiles as the known human FP receptors. “FP” means the receptor is of the class which has the highest affinity for PGF_{2α} of all the naturally occurring prostaglandins. FP refers to a known protein.

“Halogen atom” means F, Cl, Br, or I. Preferably, the halogen atom is F, Cl, or Br; more preferably Cl or F; and most preferably F.

“Halogenated heterogenous group” means a substituted heterogenous group or a substituted heterocyclic group, wherein at least one substituent is a halogen atom. Halogenated heterogenous groups can have a straight, branched, or cyclic structure. Preferred halogenated heterogenous groups have 1 to 12 carbon atoms, more preferably 1 to 6 carbon atoms, and most preferably 1 to 3 carbon atoms. Preferred halogen atom substituents are Cl and F. “Halogenated hydrocarbon group” means a substituted monovalent hydrocarbon group or a substituted carbocyclic group, wherein at least one substituent is a halogen atom. Halogenated hydrocarbon groups can have a straight, branched, or cyclic structure. Preferred halogenated hydrocarbon groups have 1 to 12 carbon atoms, more preferably 1 to 6 carbon atoms, and most preferably 1 to 3 carbon atoms. Preferred halogen atom substituents are Cl and F. The most preferred halogenated hydrocarbon group is trifluoromethyl.

“Heteroaromatic group” means an aromatic ring containing carbon and 1 to 4 heteroatoms in the ring. Heteroaromatic groups are monocyclic or fused bicyclic rings. Monocyclic heteroaromatic groups contain 5 to 10 member atoms (i.e., carbon and heteroatoms), preferably 5 to 7, and more preferably 5 to 6 in the ring. Bicyclic heteroaromatic rings contain 7 to 17 member atoms, preferably 7 to 14, and more preferably 9 or 10 in the ring. Heteroaromatic groups are unsubstituted. Preferred heteroaromatic groups include thienyl, thiazolyl, purinyl, pyrimidyl, pyridyl, and furanyl. More preferred heteroaromatic groups include thienyl, furanyl, and pyridyl. The most preferred heteroaromatic ring is thienyl.

“Heteroatom” means an atom other than carbon in the ring of a heterocyclic group or the chain of a heterogeneous group. Preferably, heteroatoms are selected from the group consisting of nitrogen, sulfur, and oxygen atoms. Groups containing more than one heteroatom may contain different heteroatoms.

“Heterocyclic group” means a saturated or unsaturated ring structure containing carbon and 1 to 4 heteroatoms in the ring. No two heteroatoms are adjacent in the ring. Heterocyclic groups are not aromatic. Heterocyclic groups are monocyclic, or are fused or bridged bicyclic ring systems. Monocyclic heterocyclic groups contain 4 to 10 member atoms (i.e., including both carbon atoms and at least 1 heteroatom), preferably 4 to 7, and more preferably 5 to 6 in the ring. Bicyclic heterocyclic groups contain 7 to 17 member atoms, preferably 7 to 14, and more preferably 9 or 10 in the ring. Heterocyclic groups are unsubstituted. Preferred heterocyclic groups include piperzyl, morpholinyl, tetrahydrofuranyl, and piperdyl.

“Heterogeneous group” means a saturated or unsaturated chain containing 1 to 18 member atoms (i.e., including both carbon and at least one heteroatom). No two heteroatoms are adjacent. Preferably, the chain contains 1 to 12 member atoms, more preferably 1 to 6, and most preferably 1 to 4. The chain may be straight or branched. Preferred branched heterogeneous groups have one or two branches, preferably one branch. Preferred heterogeneous groups are saturated. Unsaturated heterogeneous groups have one or more double bonds, one or more triple bonds, or both. Preferred unsaturated heterogeneous groups have one or two double bonds or one triple bond. More preferably, the unsaturated heterogeneous group has one double bond. Heterogeneous groups are unsubstituted.

“Monovalent hydrocarbon group” means a chain of 1 to 18 carbon atoms, preferably 1 to 12 carbon atoms. “Lower monovalent hydrocarbon group” means a monovalent hydrocarbon group having 1 to 6, preferably 1 to 4 carbon atoms. Preferred lower monovalent hydrocarbon groups include alkyl groups such as methyl, ethyl, propyl, and butyl. Monovalent hydrocarbon groups may have a straight chain or branched chain structure. Preferred branched monovalent hydrocarbon groups have one or two branches, preferably 1 branch. Monovalent hydrocarbon groups may be saturated or unsaturated. Preferred monovalent hydrocarbon groups are saturated. Unsaturated monovalent hydrocarbon groups have one or more double bonds, one or more triple bonds, or combinations thereof. Preferred unsaturated monovalent hydrocarbon groups have one or two double bonds or one triple bond; more preferred unsaturated monovalent hydrocarbon groups have one double bond. Preferred monovalent hydrocarbon groups include alkyl groups.

“Pharmaceutically acceptable” means suitable for use in a human or other mammal.

“Prostaglandin” means a fatty acid derivative which has a variety of potent biological activities of a hormonal or regulatory nature.

“Protecting group” is a group that replaces the active hydrogen of a hydroxyl moiety thus preventing undesired side reaction at the hydroxyl moiety. Use of protecting groups in organic synthesis is well known in the art. Examples of protecting groups are found in Chapter 2 Protecting Groups in Organic Synthesis by Greene, T. W. and Wuts, P. G. M., 2^{nd} ed., Wiley & Sons, Inc., 1991. Preferred protecting groups include silyl ethers, alkoxymethyl ethers, tetrahydropyranyl, tetrahydrofuranyl, esters, and substituted or unsubstituted benzyl ethers.

“Safe and effective amount” means a quantity of a prostaglandin high enough to provide a significant positive modification of the subject's condition to be treated, but low enough to avoid serious side effects (at a reasonable benefit/risk ratio).

“Selective” means having a binding or activation preference for a specific receptor over other receptors which can be quantitated based upon receptor binding or activation assays.

“Subject” means a living, vertebrate, hair- or fur-bearing animal such as a mammal (preferably human) in need of treatment.

“Substituted aromatic group” means an aromatic group wherein at least 1 (preferably 1 to 4) of the hydrogen atoms bonded to a carbon atom in the ring has been replaced with another substituent. Preferred substituents include: halogen atoms, cyano groups, monovalent hydrocarbon groups, substituted monovalent hydrocarbon groups, heterogeneous groups, aromatic groups, substituted aromatic groups, or any combination thereof. More preferred substituents include halogen atoms, monovalent hydrocarbon groups, and substituted monovalent hydrocarbon groups. Preferred substituted aromatic groups include naphthyl. The substituents may be substituted at the ortho, meta, or para position on the ring, or any combination thereof. The preferred substitution pattern on the ring is ortho or meta. The most preferred substitution pattern is ortho.

“Substituted carbocyclic group” means a carbocyclic group wherein at least 1 (preferably 1 to 4) of the hydrogen atoms bonded to a carbon atom in the ring has been replaced with another substituent. Preferred substituents include: halogen atoms, cyano groups, monovalent hydrocarbon groups, monovalent heterogeneous groups, substituted monovalent hydrocarbon groups, aromatic groups, substituted aromatic groups, or any combination thereof. More preferred substituents include halogen atoms and substituted monovalent hydrocarbon groups.

“Substituted heteroaromatic group” means a heteroaromatic group wherein 1 to 4 hydrogen atoms bonded to carbon atoms in the ring have been replaced with other substituents. Preferred substituents include: halogen atoms, cyano groups, monovalent hydrocarbon groups, substituted monovalent hydrocarbon groups, heterogeneous groups, substituted heterogeneous groups, phenyl groups, phenoxy groups, or any combination thereof. More preferred substituents include halogen atoms, halogenated hydrocarbon groups, halogenated heterogenous groups, monovalent hydrocarbon groups, and phenyl groups.

“Substituted heterocyclic group” means heterocyclic group wherein at least 1 (preferably 1 to 4) of the hydrogen atoms bonded to a carbon atom in the ring has been replaced with another substituent. Preferred substituents include: halogen atoms, cyano groups, monovalent hydrocarbon groups, substituted monovalent hydrocarbon groups, heterogeneous groups, substituted heterogeneous groups, halogenated hydrocarbon groups, halogenated heterogenous groups, aromatic groups, substituted aromatic groups, heteroaromatic groups, substituted heteroaromatic groups, phenoxy groups, or any combination thereof. More preferred substituents include halogen atoms and halogenated hydrocarbon groups. Substituted heterocyclic groups are not aromatic.

“Substituted heterogeneous group” means a heterogeneous group, wherein at least 1 of the hydrogen atoms bonded to a carbon atom in the chain has been replaced with another substituent. Preferred substituents include halogen atoms, hydroxy groups, alkoxy groups (e.g., methoxy, ethoxy, propoxy, butoxy, and pentoxy), aryloxy groups (e.g., phenoxy, chlorophenoxy, tolyloxy, methoxyphenoxy, benzyloxy, alkyloxycarbonylphenoxy, and acyloxyphenoxy), acyloxy groups (e.g., propionyloxy, benzoyloxy, and acetoxy), carbamoyloxy groups, carboxy groups, mercapto groups, alkylthio groups, acylthio groups, arylthio groups (e.g., phenylthio, chlorophenylthio, alkylphenylthio, alkoxyphenylthio, benzylthio, and alkyloxycarbonylphenylthio), aromatic groups (e.g., phenyl and tolyl), substituted aromatic groups (e.g., alkoxyphenyl, alkoxycarbonylphenyl, and halophenyl), heterocyclic groups, heteroaromatic groups, and amino groups (e.g., amino, mono- and di-alkylamino having 1 to 3 carbon atoms, methylphenylamino, methylbenzylamino, alkanylamido groups of 1 to 3 carbon atoms, carbamamido, ureido, and guanidino).

“Substituted monovalent hydrocarbon group” means a monovalent hydrocarbon group wherein at least 1 of the hydrogen atoms bonded to a carbon atom in the chain has been replaced with another substituent. Preferably 1 to 4, more preferably 1 to 3, of the hydrogen atoms bonded to a carbon atom have been replaced with other substituents. Preferred substituents include halogen atoms; substituted monovalent hydrocarbon groups; lower monovalent hydrocarbon groups such as alkyl groups (e.g., methyl, ethyl, propyl, and butyl); hydroxy groups; alkoxy groups (e.g., methoxy, ethoxy, propoxy, butoxy, and pentoxy); aryloxy groups (e.g., phenoxy, chlorophenoxy, tolyloxy, methoxyphenoxy, benzyloxy, alkyloxycarbonylphenoxy, and acyloxyphenoxy); acyloxy groups (e.g., propionyloxy, benzoyloxy, and acetoxy); carbamoyloxy groups; carboxy groups; mercapto groups; alkylthio groups; acylthio groups; arylthio groups (e.g., phenylthio, chlorophenylthio, alkylphenylthio, alkoxyphenylthio, benzylthio, and alkyloxycarbonylphenylthio); aryl groups (e.g., phenyl, tolyl, alkoxyphenyl, alkoxycarbonylphenyl, and halophenyl); heterocyclic groups; heteroaromatic groups; carbocyclic groups, heterocyclic groups, and amino groups (e.g., amino, mono- and di-alkanylamino groups of 1 to 3 carbon atoms, methylphenylamino, methylbenzylamino, alkanylamido groups of 1 to 3 carbon atoms, carbamamido, ureido, and guanidino).

### Prostaglandins Used in the Invention

This invention relates to the use of prostaglandins to treat hair loss. The prostaglandin is selected from the group consisting of oximyl- and hydroxylamino-prostaglandins having the structure:

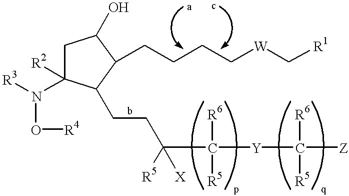

and pharmaceutically acceptable salts and hydrates of the structure above; biohydrolyzable amides, esters, and imides of the structure above; optical isomers, diastereomers, and enantiomers of the structure above; and combinations thereof.

W is selected from the group consisting of O, NH, S, S(O), S(O)₂, and —(CH₂)ₘ—, wherein m is 0 to 3. Preferably, W is selected from the group consisting of O and —(CH₂)ₘ—, and more preferably, W is —CH₂—.

X is selected from the group consisting of NHR⁸, OR⁸, SR⁹, and S(O)R⁹. Preferably, X is OR⁸.

Y is selected from the group consisting of a bond, an oxygen atom, a sulfur atom, NHR⁸, S(O), and S(O)₂; with the proviso that when Y is NHR⁸, no carbon atom in R⁸ is bonded to more than one heteroatom. Preferably, Y is selected from the group consisting of a bond, an oxygen atom, and NHR⁵ More preferably, Y is a bond or an oxygen atom.

Z is selected from the group consisting of H, CH₃, a carbocyclic group, a heterocyclic group, a substituted carbocyclic group, a substituted heterocyclic group, an aromatic group, a heteroaromatic group, a substituted aromatic group, and a substituted heteroaromatic group. Z is preferably selected from the group consisting of aromatic, heteroaromatic, substituted aromatic, and substituted heteroaromatic groups. More preferably, the aromatic, heteroaromatic, substituted aromatic, and substituted heteroaromatic groups are monocyclic and have 6 member atoms in the ring. Still more preferably, Z is selected from the group consisting of thienyl and phenyl. Preferably, when Y is S, S(O), or S(O)₂ and Z is H, q is at least 1.

R¹ is selected from the group consisting of CO₂H, CO₂R⁷, C(O)NHOH, S(O)₂R⁷, C(O)NHS(O)₂R⁷, and tetrazole. Preferably, R¹ is selected from the group consisting of CO₂H, C(O)NHOH, CO₂R⁷, C(O)NHS(O)₂R⁷, and tetrazole, More preferably, R¹ is selected from the group consisting of CO₂H, CO₂R⁷, and C(O)NHOH.

R² is hydrogen, and R³ is hydrogen or a lower monovalent hydrocarbon group, with the proviso that alternatively, R² and R³ may form a covalent bond (i.e., the oximyl structure).

R⁴ is a hydrogen atom, a monovalent hydrocarbon group, a heterogeneous group, a carbocyclic group, heterocyclic group, an aromatic group, a heteroaromatic group, a substituted monovalent hydrocarbon group, a substituted heterogeneous group, a substituted carbocyclic group, a substituted heterocyclic group, a substituted aromatic group, or a substituted heteroaromatic group. Preferably, R⁴ is selected from the group consisting of a hydrogen atom and a monovalent hydrocarbon group of 1 to 8 carbon atoms. More preferably, R⁴ is a hydrogen atom or a lower monovalent hydrocarbon group. Still more preferably, R⁴ is a hydrogen atom or a methyl group. Most preferably, R⁴ is a hydrogen atom.

Each R⁵ is independently selected from the group consisting of H, CH₃, and C₂H₅. Preferably, R⁵ is selected from the group consisting of H and CH₃; more preferably, R⁵ is H.

Each R⁶ is independently selected from the group consisting of H, CH₃, C₂H₅, OR⁸, and NHR⁸. Preferably, R⁶ is selected from the group consisting of H, CH₃, C₂H₅, and OR⁸. More preferably, R⁶ is H or CH₃.

R⁷ is selected from the group consisting of monovalent hydrocarbon groups, heterogeneous groups, aromatic groups, heteroaromatic groups, monocyclic carbocyclic groups, monocyclic heterocyclic groups, substituted monovalent hydrocarbon groups, substituted aromatic groups, and substituted heteroaromatic groups. R⁷ preferably contains 1 to 8 carbon atoms. R⁷ is more preferably selected from the group consisting of methyl, ethyl, and isopropyl groups.

Each R⁸ is independently selected from the group consisting of a hydrogen atom, an acyl group, a monovalent hydrocarbon group, a substituted monovalent hydrocarbon group, a heterogeneous group, a substituted heterogeneous group, a carbocyclic group, a substituted carbocyclic group, and heterocyclic group, a substituted heterocyclic group, an aromatic group, a substituted aromatic group, a heteroaromatic group, and a substituted heteroaromatic group. R⁸ is preferably a hydrogen atom.

Each R⁹ is independently selected from the group consisting of a monovalent hydrocarbon group, a substituted monovalent hydrocarbon group, a heterogeneous group, a substituted heterogeneous group, a carbocyclic group, a substituted carbocyclic group, and heterocyclic group, a substituted heterocyclic group, an aromatic group, a substituted aromatic group, a heteroaromatic group, and a substituted heteroaromatic group.

The subscript p is an integer with a value of 0 to 6, preferably 1 to 5, and the subscript q is an integer with a value of 0 to 5, with the proviso that (p+q)=1 to 5. When Y is a bond and p is 0, q is preferably 2 or 3.

Bonds a, b, and c are each independently selected from the group consisting of a single bond, a cis double bond, and a trans double bond. Preferably, bond a is a single bond or a cis double bond. Preferably, bond b is a single bond or a trans double bond. Preferably, bond c is a single bond.

Examples of prostaglandins having the formula above are shown in Table 1.

TABLE 1
Examples of Prostaglandins Suitable for Component A

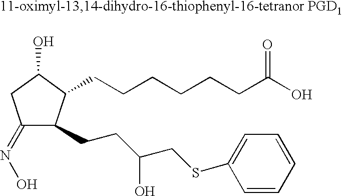

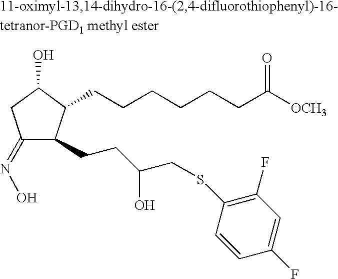

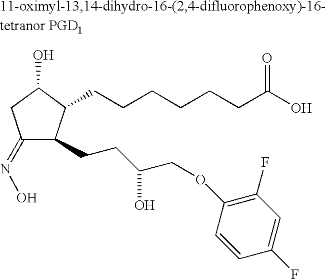

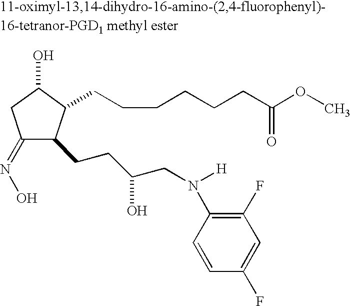

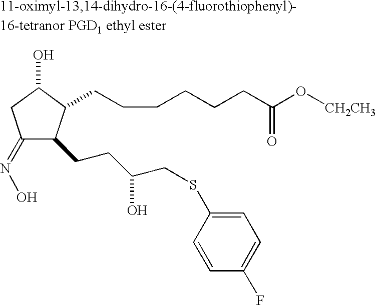

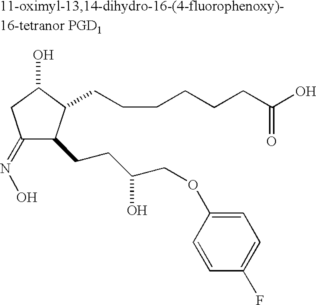

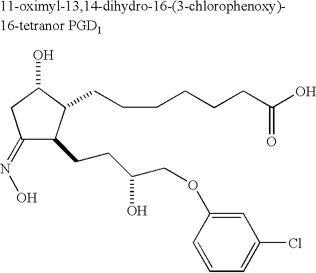

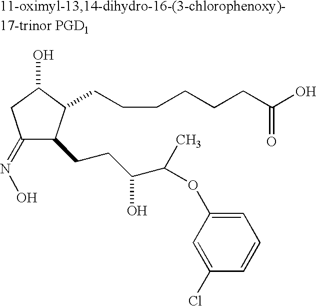

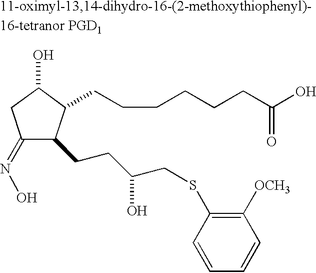

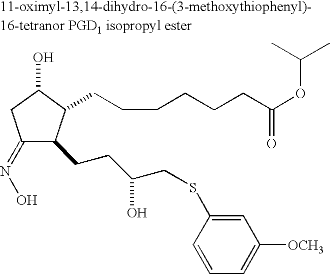

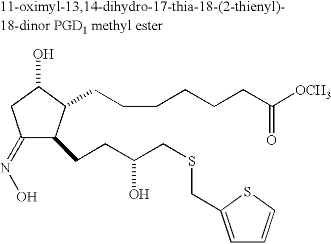

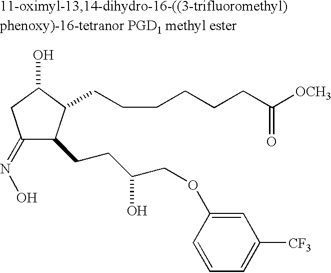

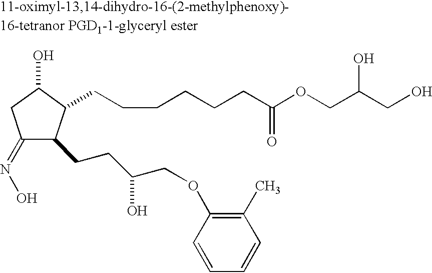

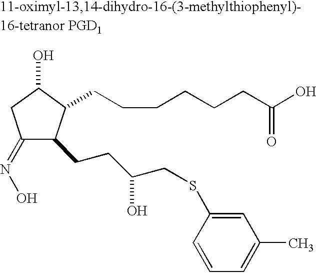

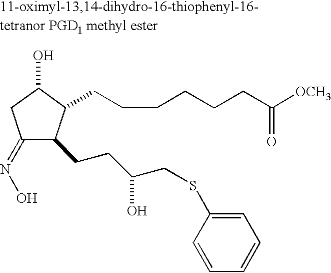

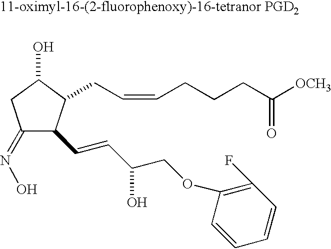

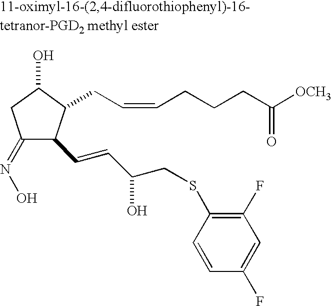

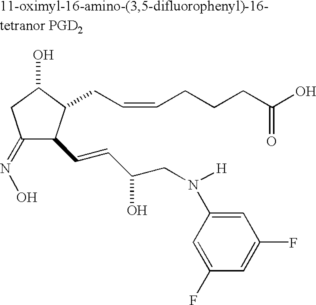

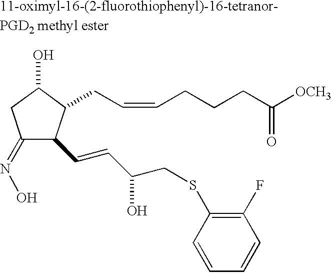

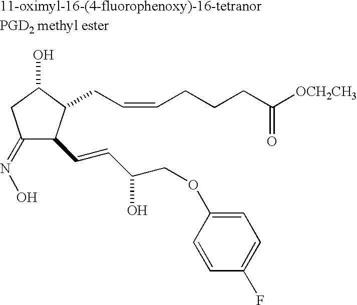

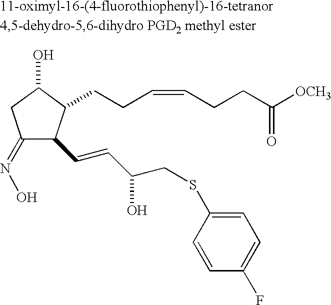

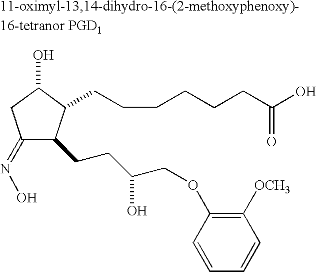

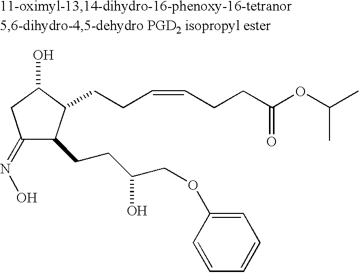

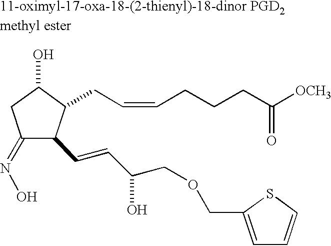

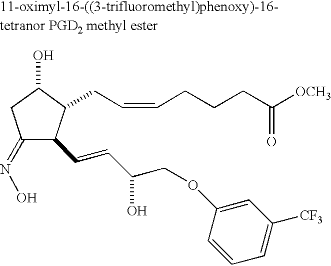

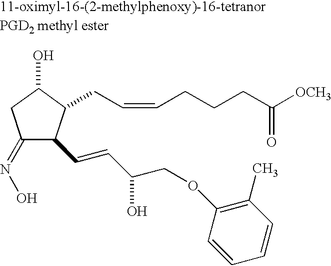

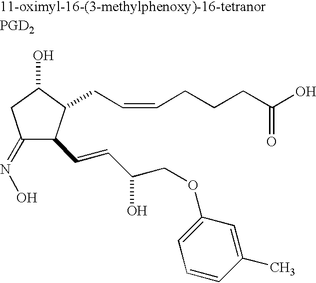

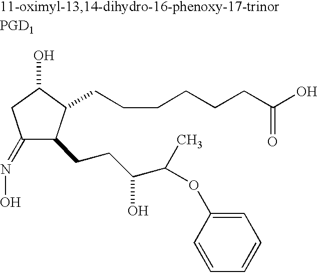

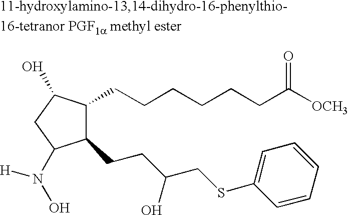

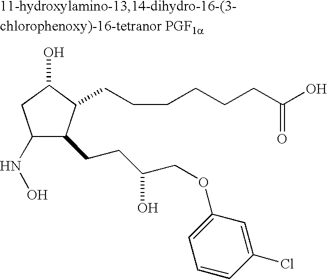

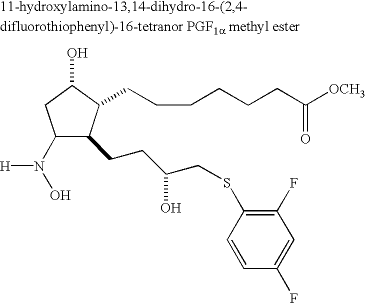

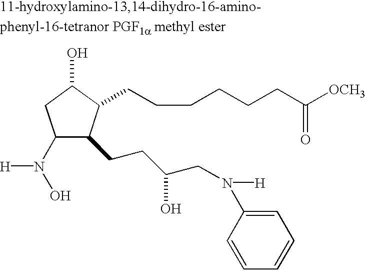

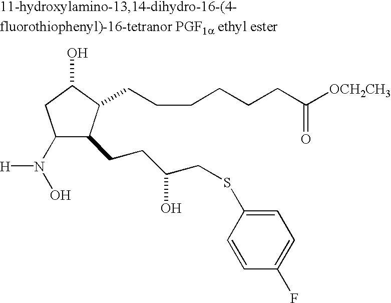

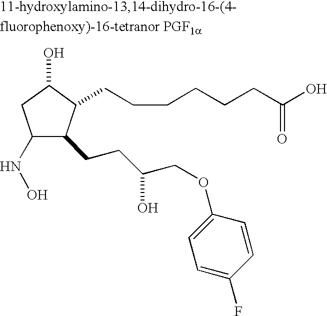

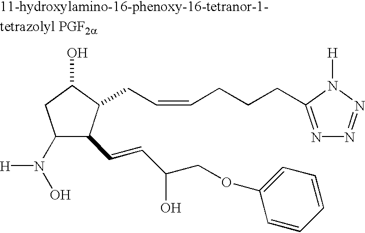

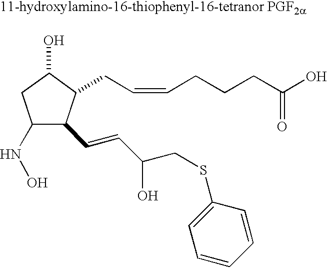

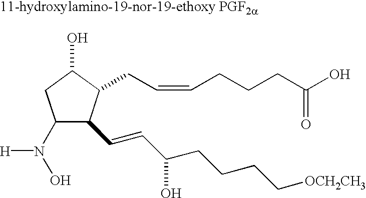

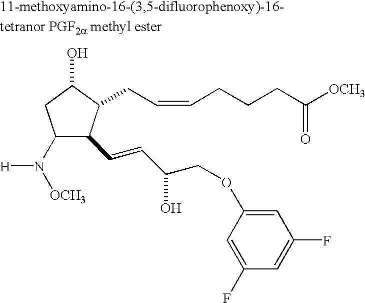

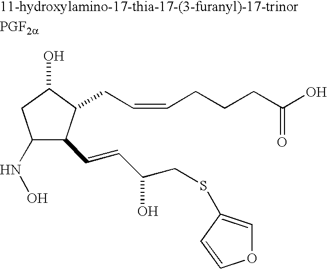

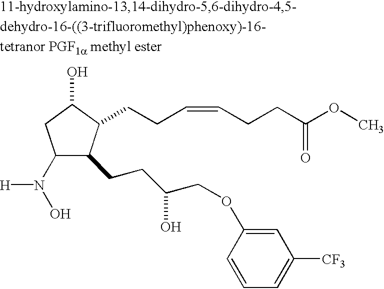

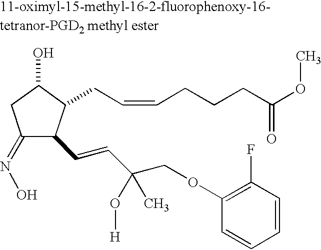

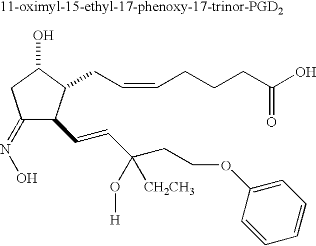

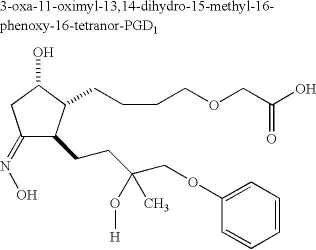

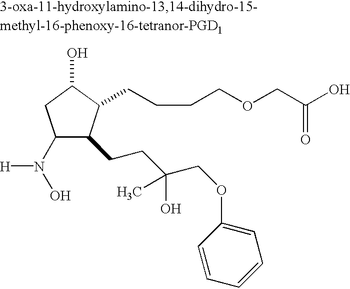

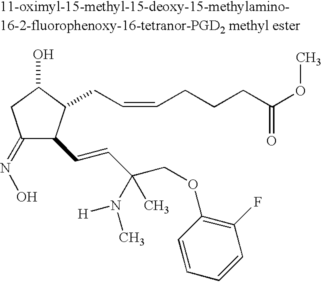

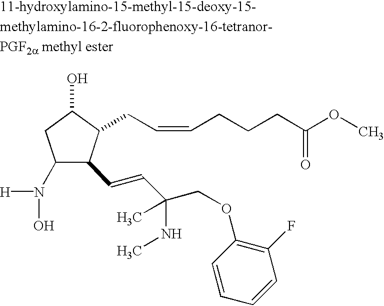

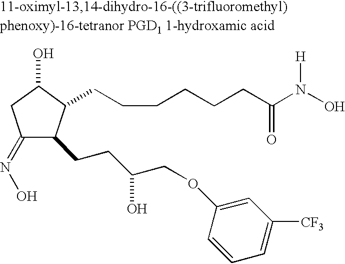

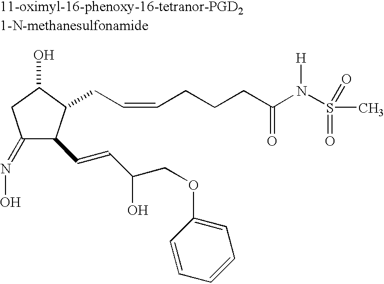

Of the prostaglandins in Table 1, 11-oximyl-16-((3-trifluoromethyl)phenoxy)-16-tetranor PGD₂ methyl ester, 11-oximyl-13,14-dihydro-16-phenoxy-16-tetranor 5,6-dihydro-4,5-dehydro PGD₂ isopropyl ester, and 11-oximyl-16-phenoxy-16-tetranor PGD₂ are preferred.

When Y is a bond and q is 0, the prostaglandin will have the formula:

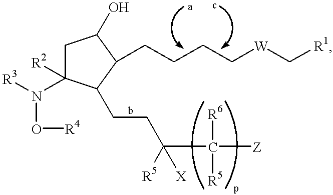

wherein R¹, W, R², R³, R⁴, R⁵, X, R⁶, Z, p, bond a, and bond b are as described above. Examples of suitable prostaglandins having this formula are shown in Table 2.

TABLE 2
Examples of Prostaglandins Suitable for Component A

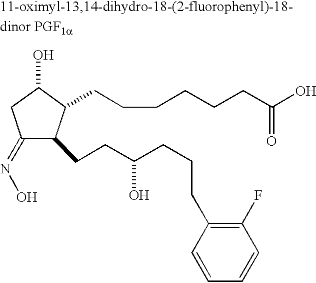

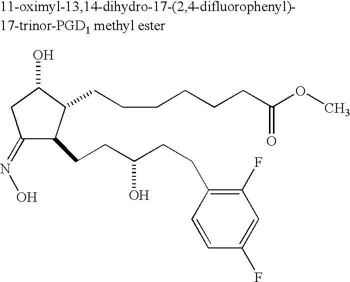

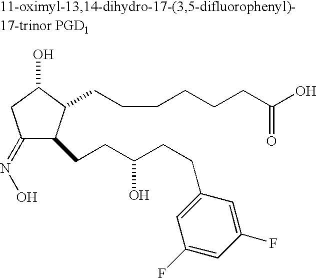

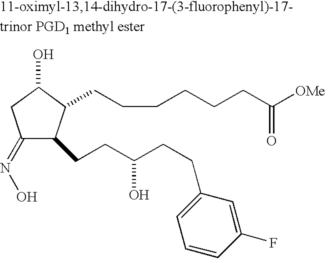

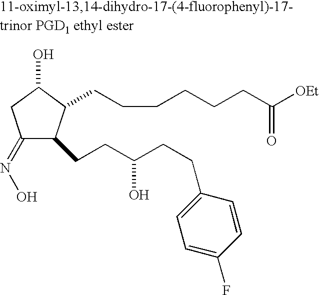

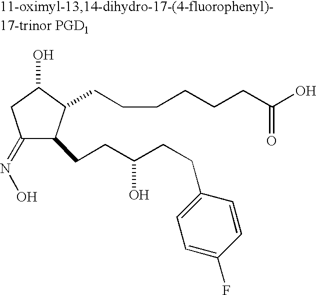

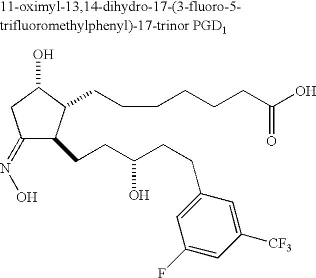

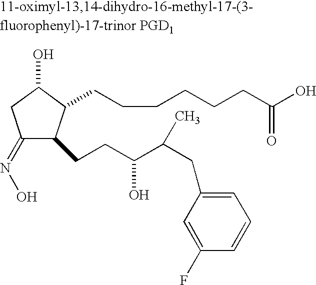

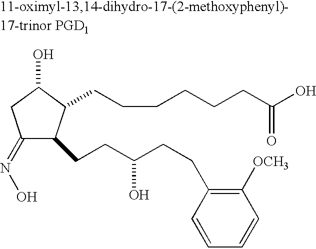

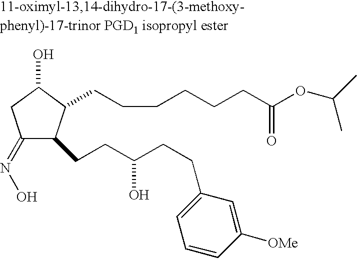

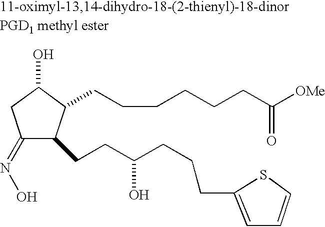

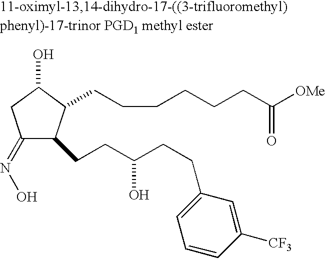

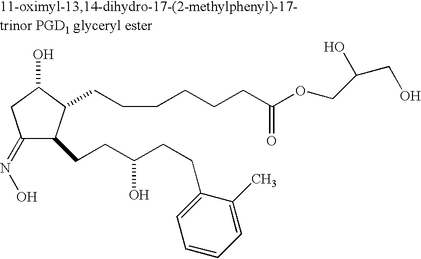

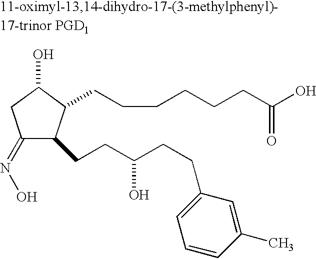

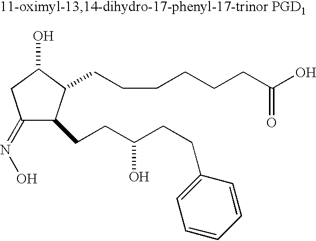

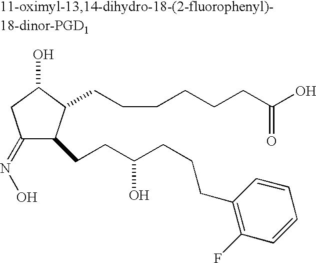

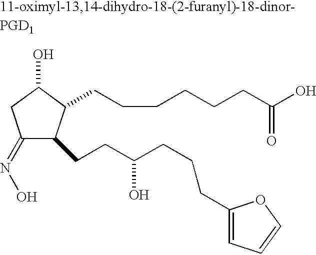

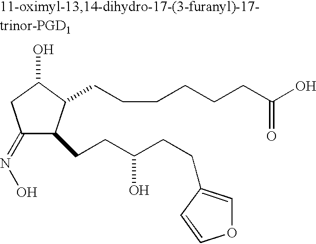

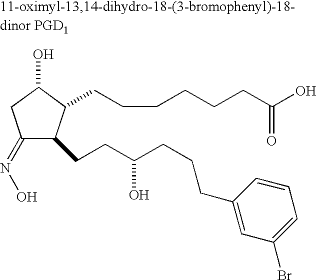

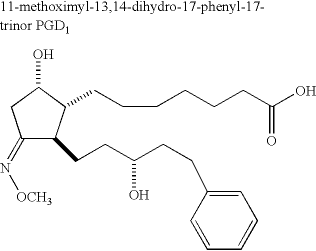

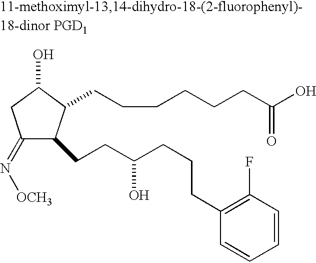

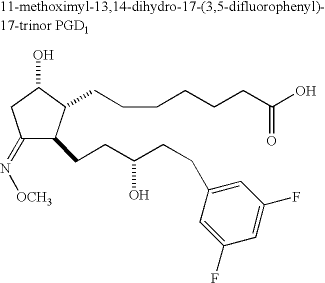

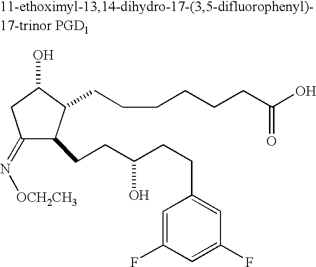

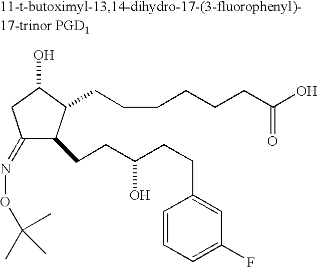

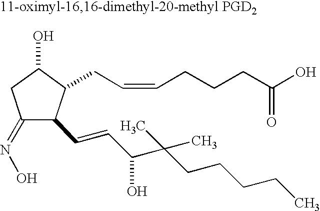

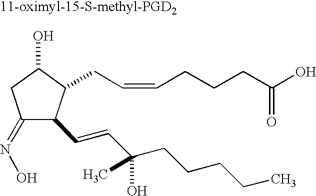

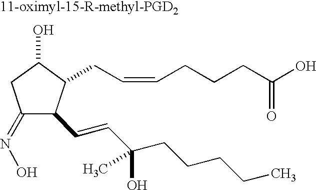

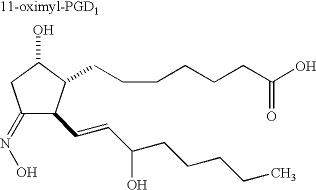

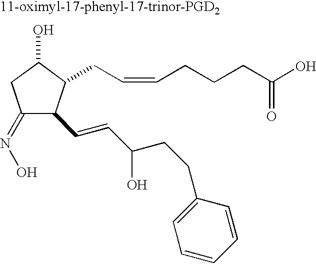

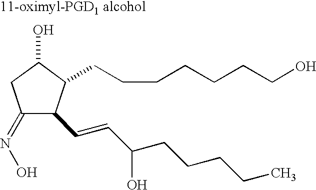

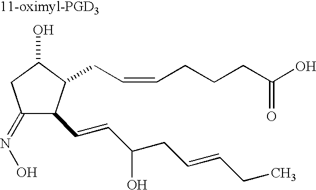

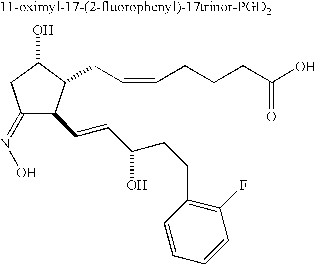

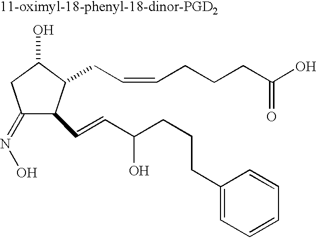

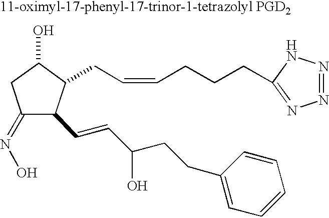

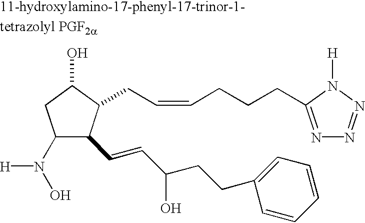

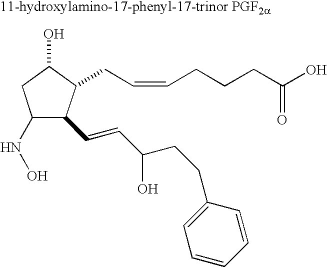

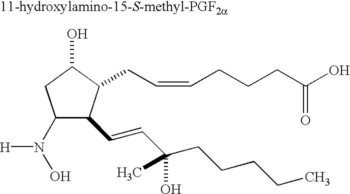

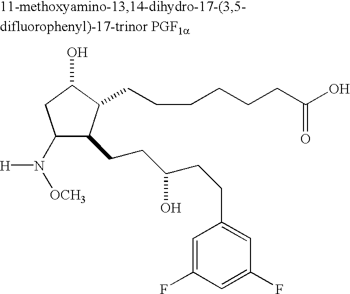

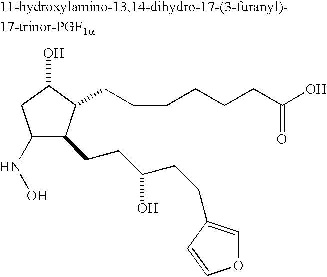

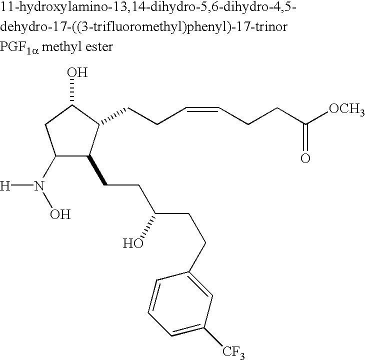

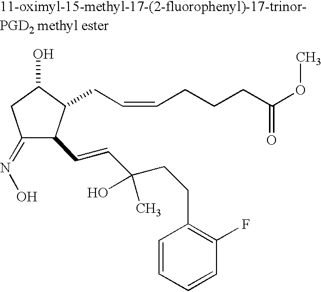

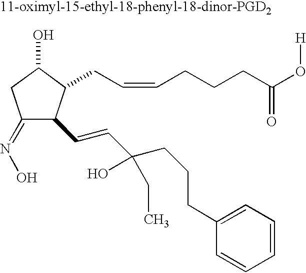

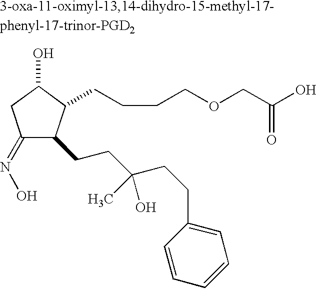

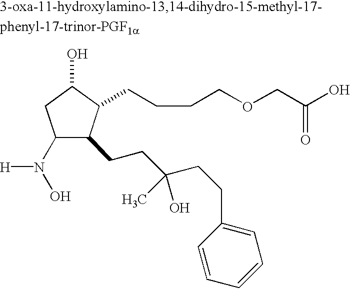

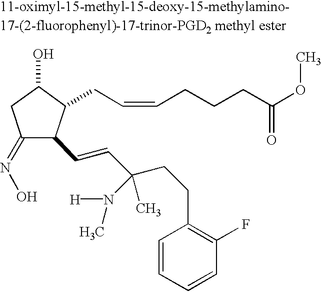

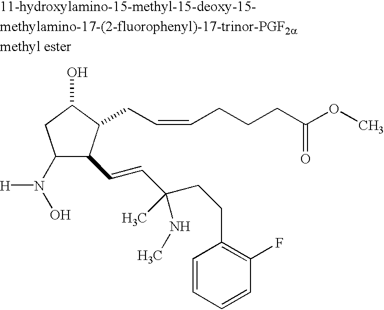

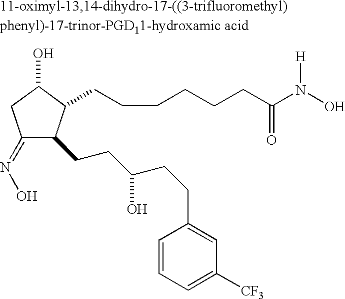

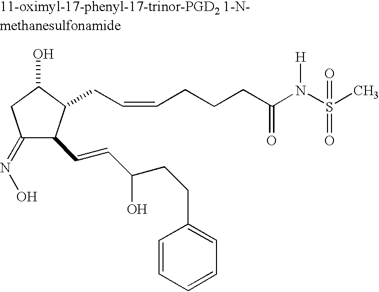

(In the table above, Me represents a methyl group, and Et represents an ethyl group.)

Of the compounds in Table 2, 11 -oximyl-PGD₂ and 11-hydroxylamino-15-S-methyl-PGF_{2α} are preferred.

Even though the some of the prostaglandins having the structures above are more structurally similar to PGD analogs than PGF analogs, the above prostaglandins selectively activate the FP receptor and do not activate the DP receptor. Without wishing to be bound by theory, it is believed that the functionality (shown below) at the C11 position in the structures above imparts the selectivity to bind with the FP receptor.

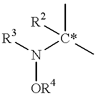

Therefore, any FP agonist containing this functionality, wherein C* is one of the carbon atoms in the cyclopentyl ring, that selectively activates the FP receptor is also suitable to use in this invention. Preferably, C* is the carbon atom at the C11 position.

Prostaglandins suitable for use in this invention can be made using conventional organic syntheses. Preferred syntheses are exemplified by the following two general reaction schemes:

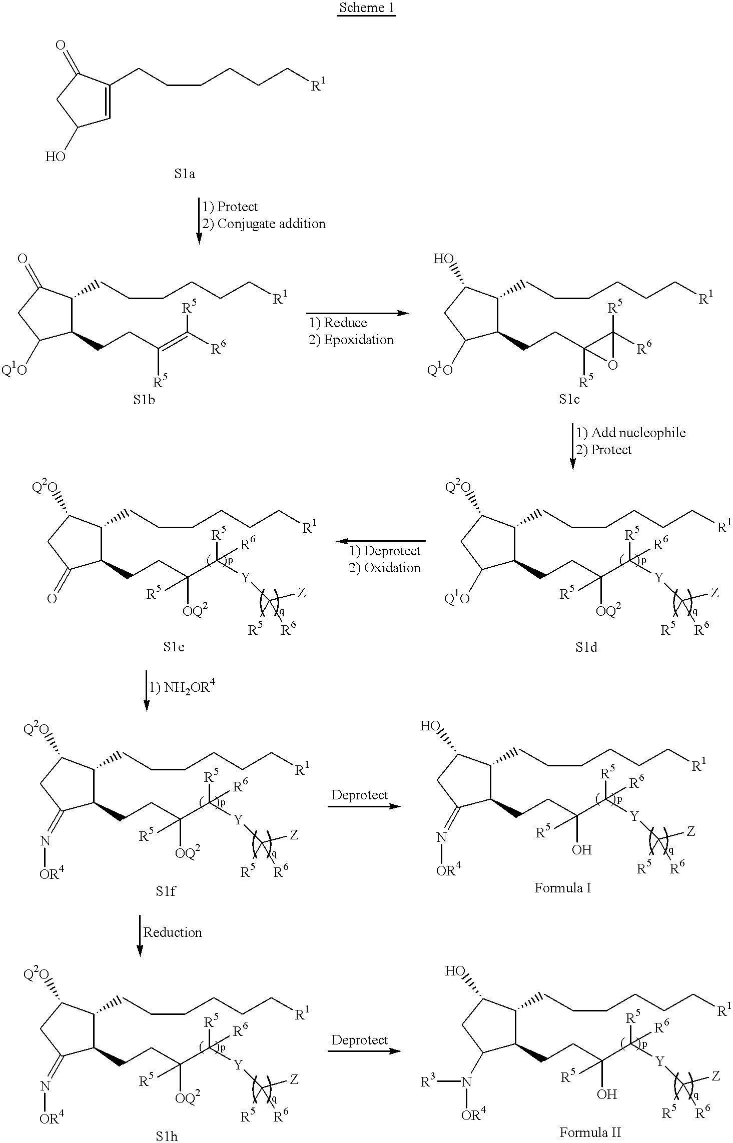

In Scheme 1, R¹, R², R³, R⁴, R⁵, R⁶, X, Y, p, q, and Z are as defined above. Q¹ is a silyl-functional protecting group. Q² is a protecting group. The methyl 7(3-(R)-hydroxy-5-oxo-1-cyclopent-1-yl) heptanoate (S1a) depicted as starting material for Scheme 1 is commercially available (such as from Sumitomo Chemical or Cayman Chemical).

In the above Scheme 1, methyl 7-(3-(R)-hydroxy-5-oxo-1-cyclopent-1-yl) heptanoate (S1a) is reacted with a silylating agent and base in a solvent that will allow the silylation to proceed. Preferred silylating agents include tert-butyldimethylsilyl chloride and tert-butyldimethylsilyl trifluoromethanesulfonate. The most preferred silylating agent is tert-butyldimethylsilyl trifluoromethanesulphonate. Preferred bases include triethylamine, trimethylamine, and 2,6-lutidine. More preferred bases include triethylamine and 2,6-lutidine. The most preferred base is 2,6-lutidine. Preferred solvents include halogenated hydrocarbon solvents with dichloromethane being the most preferred solvent. The reaction is allowed to proceed at a temperature preferably of −100° C. to 100° C., more preferably −80° C. to 80° C., and most preferably −70° C. to 23° C.

The resulting silylated compound is isolated by methods known to those of ordinary skill in the art. Such methods include extraction, solvent evaporation, distillation, and crystallization. Preferably, the silyl ether is purified after isolation by distillation under vacuum.

The silylated compound is then reacted with the cuprate generated via Grignard formation of the appropriate alkenyl bromide as disclosed, for example, in the following references: H. O. House et. al., “The Chemistry of Carbanions: A Convenient Precursor for the Generation of Lithium Organocuprates”, J. Org. Chem. Vol. 40 (1975) pp. 1460-69, and P. Knochel et. al., “Zinc and Copper Carbenoids as Efficient and Selective a′/d′ Multicoupling Reagents”, J. Amer. Chem. Soc. Vol. 111 (1989) p. 6474-76. Preferred alkenyl bromides include 4-bromo-1-butene, 4-bromo-1-butyne, 4-bromo-2-methyl-1-butene, and 4-bromo-2-ethyl-1-butene. The most preferred alkenyl bromide is 4-bromo-1-butene. Preferred solvents include ethereal solvents, of which diethyl ether and tetrahydrofuran are preferred. The most preferred solvent is tetrahydrofuran. The Grignard reagent is allowed to form at a temperature of 80° C. to 23° C., more preferably 80° C. to 30° C., and most preferably 75° C. to 65° C. The reaction time is preferably 1 hour to 6 hours, more preferably 2 to 5 hours, and most preferably 3 to 4 hours.

Once the Grignard reagent is formed, the cuprate is generated from the alkenyl magnesium species. The temperature range for cuprate formation is −100° C. to 0° C., preferably −80° C. to −20° C., and more preferably −75° C. to −50° C. The preferred reaction time is 30 to and 6 hours, more preferably 45 minutes to 3 hours, and most preferably 1 to 1.5 hours.

The compound depicted as S1b is isolated by methods known to one of ordinary skill in the art. Such methods include extraction, solvent evaporation, distillation, and crystallization. Preferably, S1b is purified by flash chromatography on silica gel (Merck, 230-400 mesh) using 10% EtOAc/hexanes as the eluent.

S1b is then reacted with a hydride reducing agent and a polar, protic solvent to give the C₉ alcohol. Preferred reducing agents include lithium aluminum hydride, sodium borohydride, and L-selectride. More preferred reducing agents include sodium borohydride, and L-selectride. The most preferred reducing agent is sodium borohydride. Preferred solvents include methanol, ethanol, and butanol. The most preferred solvent is methanol. The reduction is carried out at a temperature of −100° C. to 23° C., preferably −60° C. to 0° C., and most preferably −45° C. to −20° C.

The resulting alcohol of S1b is isolated by methods known to one of ordinary skill in the art. Such methods include extraction, solvent evaporation, distillation, and crystallization. Preferably, the alcohol is purified by flash chromatography on silica gel (Merck, 230-400 mesh) using 20% EtOAc/hexanes as the eluent.

The alcohol can be protected as described above. The protected or unprotected alcohol is then treated with meta-chloroperbenzoic acid in a halocarbon solvent to provide the novel epoxide intermediate depicted as S1c. Preferred halocarbon solvents include dichloromethane, dichloroethane, and chloroform. More preferred halocarbon solvents are dichloromethane and dichloroethane. The most preferred halocarbon solvent is dichloromethane.

The compound depicted as S1c is isolated by methods known to one of ordinary skill in the art. Such methods include extraction, solvent evaporation, distillation, and crystallization. Preferably, S1c is purified by flash chromatography on silica gel (Merck, 230-400 mesh) using 20% EtOAc/hexanes as the eluent.

The intermediate epoxide depicted as S1c can be reacted with a variety of oxygen, sulfur and nitrogen containing nucleophiles as disclosed, for example, in J. G. Smith, “Synthetically Useful Reactants of Epoxides”, Synthesis (1984) p. 629-656, to provide the C₁₁-protected 13,14-dihydro-15-substituted-16-tetranor prostaglandin F_{1α} derivatives.

With sulfur nucleophiles, the reaction is carried out at a temperature of preferably 80° C. to 0° C., more preferably 80° C. to 20° C., and most preferably 80° C. to 50° C. Preferred bases for the reaction include triethylamine, N,N diisopropylethylamine, and trimethylamine. The most preferred base is triethylamine. Preferred solvents for the reaction are aromatic hydrocarbon solvents. Preferred solvents include xylenes, toluene, and benzene. The most preferred solvent is benzene. With nitrogen and oxygen nucleophiles, preferred solvents include ethereal solvents and polar, protic solvents. More preferred ethereal solvents include diethyl ether, dibutyl ether and tetrahydrofuran. The most preferred ethereal solvent is tetrahydrofuran. More preferred polar, protic solvents include ethyl alcohol, methyl alcohol, and tert-butyl alcohol. The most preferred polar, protic solvent is ethyl alcohol.

The ring-opening process with nitrogen and oxygen nucleophiles can be catalyzed with Lewis acids. Preferred Lewis acids include magnesium perchlorate, trimethylsilyl trifluoromethanesulphonate, and trimethylaluminum. The most preferred Lewis acid is magnesium perchlorate. The reaction is carried out at a temperature of 80° C. to 23° C., preferably 80° C. to 40° C., and more preferably 80° C. to 70° C.

The selective protection of C-9 and C-15 can be accomplished by methods known to one of ordinary skill in the art. Preferred protecting groups include, but are not limited to acylating agents, alkylating agent, and carbonate forming agents. The most preferred protecting group is acetyl. Preferred solvents include halohydrocarbon and amine solvents. The most preferred is pyridine. Preferred reagents include acetyl halides and acetic anhydride. The most preferred is acetic anhydride. The temperature range for the reaction is −100° C. to 100° C., preferably −10° C. to 40° C., and more preferably −5° C. to 40° C. The preferred reaction time is 1 to 48 hours, preferably 6 to 24 hours.

The compound depicted as S1d is isolated by methods known to one of ordinary skill in the art. Such methods include extraction, solvent evaporation, distillation, and crystallization. Preferably, S1d is purified by flash chromatography on silica gel (Merck, 230-400 mesh) using 10% EtOAc/hexanes as the eluent.

The resulting C-11 ether on compound S1d is deprotected using using a fluoride or its equivalent. The deprotection reagents include tetrabutyl ammonium fluoride, hydrogen fluoride in pyridine, potassium fluoride, and treatment with strong acid. Preferred is HF/pyridine. The temperature range is −100° C. to 50° C. The preferred temperature range is −50° C. to 30° C. The most preferred is −20° C. to 10° C. The preferred solvents are THF, acetonitrile, and Et₂O. Most preferred is acetonitrile. The compound is isolated by methods known to one of ordinary skill in the art. Such methods include extraction, solvent evaporation, distillation, and crystallization. Preferably the compound is purified by flash chromatography on silica gel (Merck, 230-400 mesh) using 20% EtOAc/hexanes as the eluent.

Compound S1e is produced by the oxidation of the C11 alcohol to give the ketone. The oxidation can be accomplished by, for example, Swern, Jones, PCC, PDC. The most preferred is PCC. The most preferred solvent is dichloromethane. The preferred reaction temperature is −30° C. to 100° C. The most preferred is 0° C. to 50° C. Compound S1e is isolated by methods known to one of ordinary skill in the art. Such methods include extraction, solvent evaporation, distillation, and crystallization. Preferably the compound is purified by filtering through FLUORISLTM or silica gel and solvent evaporation.

Compound S1f is formed by the reaction of NH₂OR₄ in buffered solution of solvents. The preferred buffer is sodium acetate. The preferred solvent ratio is 3:1:1 (methanol:dioxane:water). The preferred temperature range is −20° C. to 100° C. The compound depicted as S1f is isolated by methods known to one of ordinary skill in the art. Such methods include extraction, solvent evaporation, distillation, and crystallization. Preferably, S1f is purified by flash chromatography on silica gel (Merck, 230-400 mesh) using 10% EtOAc/hexanes as the eluent.

Deprotection of S1f is accomplished by methods known to one of ordinary skill in the art and yields compounds of Formula I.

Reduction of the oxime of S1f gives the compound S1h as the hydroxyl amine. The reduction is accomplished by treatment with sodium cyanoborohydride. The preferred solvent is methanol. The preferred temperature range is −100° C. to 100° C. Deprotection of S1h is accomplished by methods known to one of ordinary skill in the art and yields compounds of Formula II.

Alternatively, the prostaglandins used in this invention can be prepared according to reaction schemes 2-1, 2-2, 2-3, and 2-4. In schemes 2-1 through 2-4, R¹, R², R³, R⁴, R⁵, R⁶ W, X, and Z are as defined above. Q¹ and Q² are protecting groups.

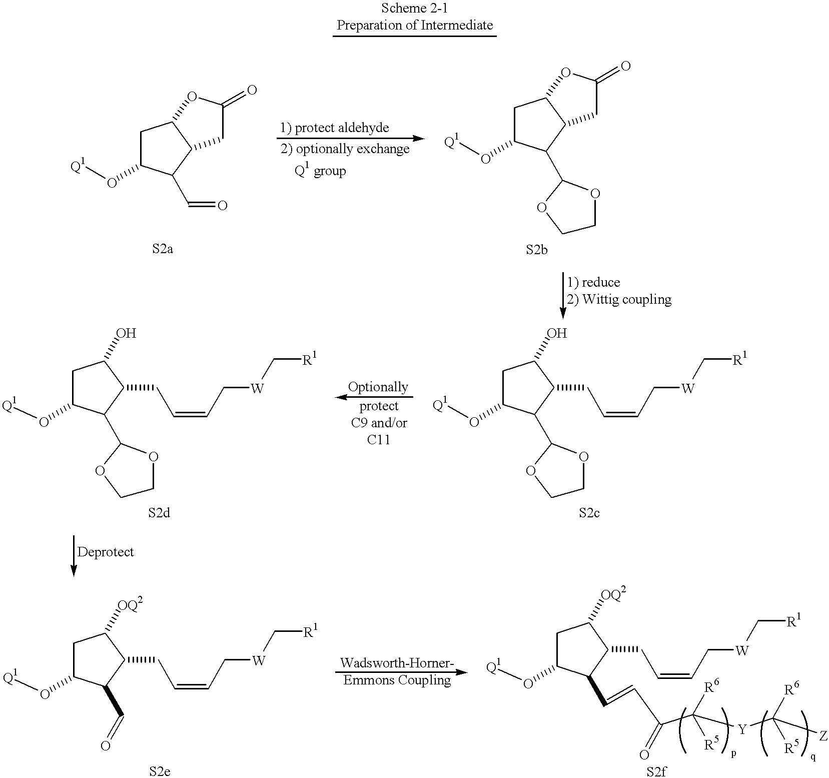

In scheme 2-1, intermediate S2f is prepared. The Corey Aldehyde (S2a) depicted as starting material for Scheme 2-1 is commercially available (such as from Aldrich Chemical or Cayman Chemical). The Corey Aldehyde (S2a) is commercially-available with a protecting group Q¹ attached to the alcohol. Q¹ can be either a silyl group or an ester group. The preferred protecting groups for Q¹ include tert-butyldimethylsilyl, acetate, benzoate, and para-phenyl benzoate. The most preferred protecting group for Q¹ is tert-butyldimethylsilyl.

The Corey aldehyde (S2a) is first reacted with an aldehyde protecting group to make a ketal or acetal. Examples of this type of protection are found in Greene and Wuts, Protecting Groups in Organic Synthesis, 2d ed., Wiley & Sons, N.Y. 1991. In this case, especially preferred are cyclic ketals and acetals. The aldehyde (S2a) is reacted with the appropriate 1,2-diol and a suitable acidic catalyst. The solvent can be the diol, and an anhydrous solvent, such as ether or dichloromethane. Particularly useful is 1,2-bis-TMS ethylene glycol to effect this transformation in ether at room temperature.

The ketal-protected S2a may then undergo a routine of protection or deprotection if desired, to exchange the Q¹ group for a more suitable one, using procedures known in the art. Particularly useful is the exchange of a silyl group for an acyl group, and vice versa. Also useful is the exchange of a silyl or acyl group for an o-bromo-benzyl ether group.

The compound (S2b) is then subjected to a DIBAL reduction to make the hemiacetal. This intermediate is not isolated but reacted as soon as possible with a Wittig salt to form an alkene (S2c). Particularly preferred Wittig salts are derived from omega bromo- four to five carbon straight chain carboxcyclic acids and 3-oxo-carboxcyclic acids. These are conveniently combined with triphenylphosphine in a suitable solvent to form the reactive Wittig salts. Other preferred reagents include straight chain omega-bromo tetrazoles and primary nitriles.

The compound (S2c) is not isolated, but reacted crude with diazomethane in diethyl ether or, preferably, with TMS diazomethane in methanol to give S2d. In addition, a suitable protecting group Q² may be placed on the C₉ alcohol at this time. The compound S2d is isolated by methods known to one of ordinary skill in the art. Such methods include extraction, solvent evaporation, distillation, and crystallization. Preferably, it is purified by flash chromatography on silica gel (Merck, 230-400 mesh) using 10% EtOAc/hexanes as the eluent.

The compound (S2d) is then optionally reduced at C-5,6 to give the saturated alpha chain of the prostaglandin, if desired, or taken on without reduction. The cyclic ketal is removed with acid or acidic ion exchange resin in a suitable solvent to give the free aldehyde. Preferred solvents include tetrahydrofuran/water mixtures.

The resulting aldehyde (S2e) is not isolated but reacted with ketone-stabilized phosphonium salts. These are generally referred to as “Wadsworth-Horner-Emmons” reagents. This reaction requires a mild base. Examples of suitable bases include sodium carbonate or triethyl arnine. The ketone (intermediate S2f) is purified by methods known to one of ordinary skill in the art. Such methods include extraction, solvent evaporation, distillation, and crystallization. Preferably, the ketone (intermediate S2f) is purified by flash chromatography on silica gel (Merck, 230-400 mesh) using 20% EtOAc/hexanes as the eluent.

The ketone (intermediate S2f) can be reacted in three ways as shown in schemes 2-2, 2-3, and 2-4.

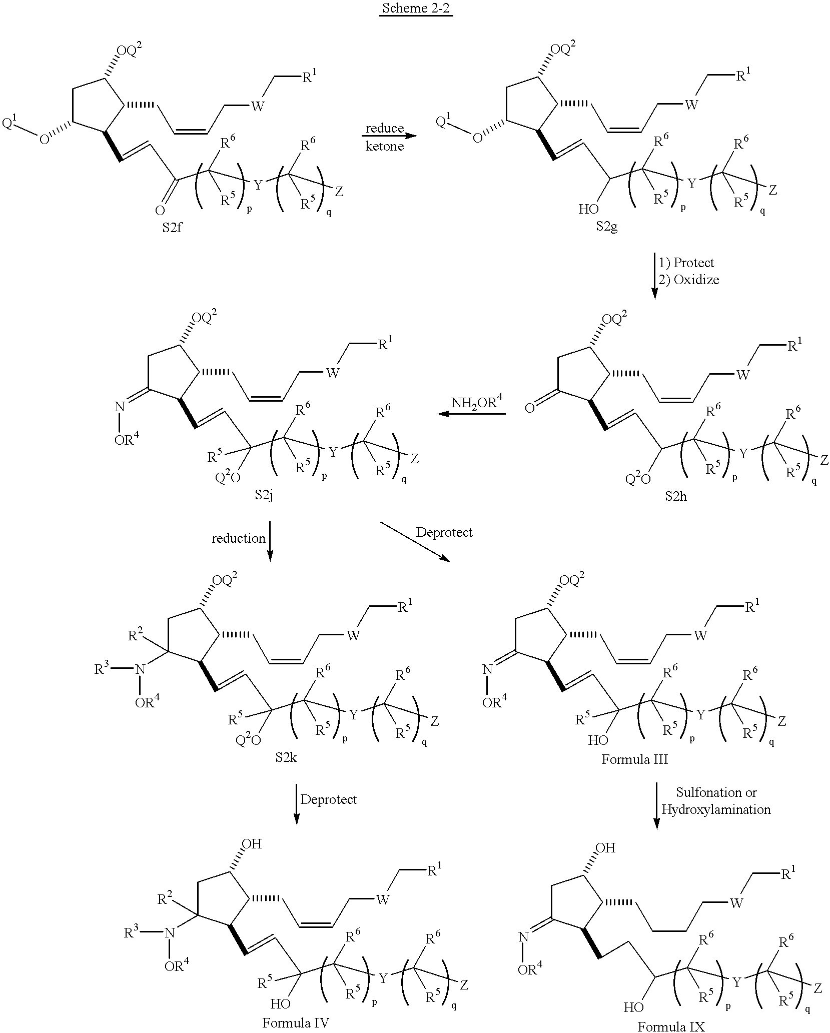

In scheme 2-2, reduction of the ketone with a reducing agent such as the Luche reagent, effects an alcohol at C-15, as illustrated by S2g. At this point, the alcohols of S2g at C-9 and C-15 may be protected, if needed or desired. If so, the alcohols can be protected as described previously herein. The S2g compound containing protected or unprotected alcohols is then treated with a deprotecting agent to release selectively Q¹ on C-11. Examples of such selective deprotection reactions are given in Greene and Wuts.

Alternatively, when Q¹ is the o-bromobenzyl ether, reduction of the bromine with a radical reducing agent such as (n-butyl)₃SnH will cause the radical-induced oxidation of C-11 to the ketone without needing protection.

Compounds of the type S2h can be converted into compounds of Formula III and Formula IV.

Compounds of Formula IX can be made from sulfonation or hydroxylamination of compounds of Formula III. In Formula IX, R¹ is a sulfonamide group or a hydroxamic acid group.

These compounds are isolated by methods known to one of ordinary skill in the art. Such methods include extraction, solvent evaporation, distillation, and crystallization.

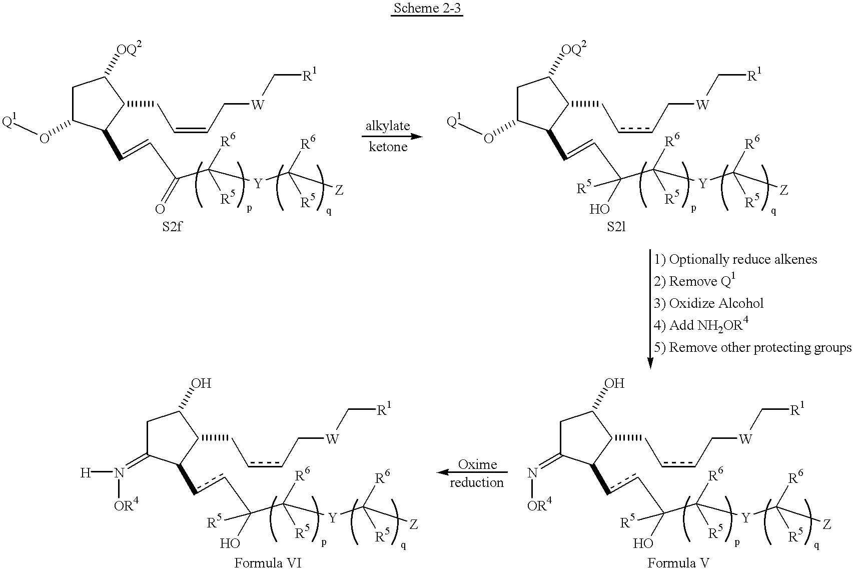

The ketone (S2f) can also be converted into compounds of the type S21. This occurs by the addition of suitable nucleophile to the ketone (S2f). Examples of nucleophiles include methyl magnesium bromide. Using substantially the same techniques described above, the compounds of the type S21 can be converted into compounds of Formula V, and compounds of Formula V can be converted into compounds of Formula VI.

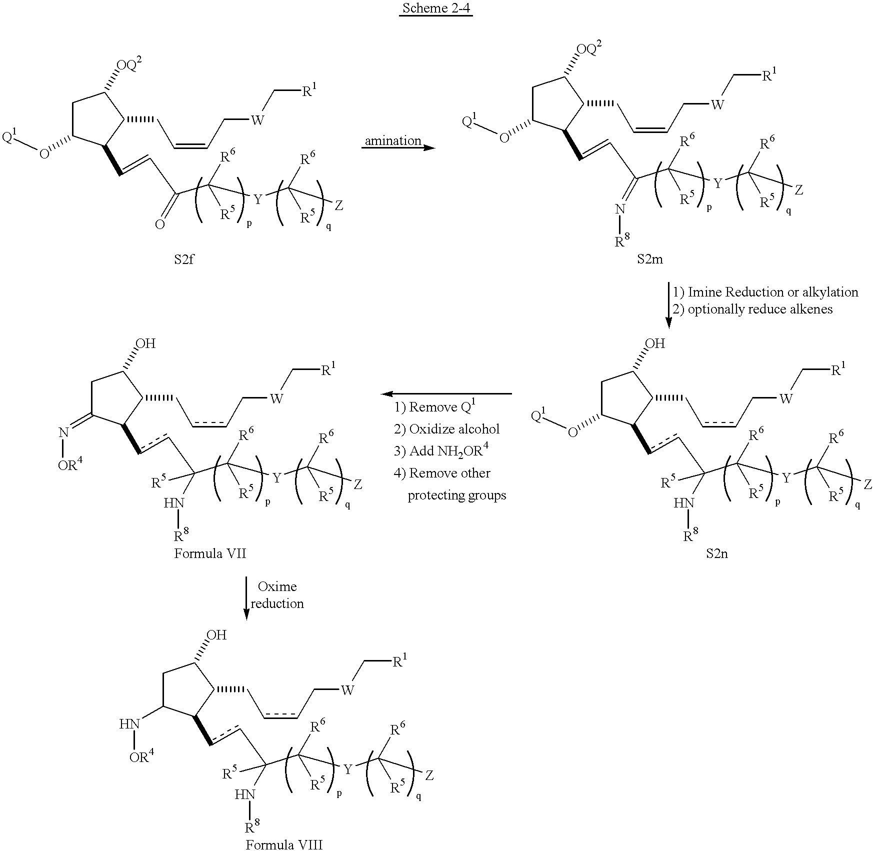

Compounds of the type S2f can also be reacted to give compounds of the type S2m by reacting the ketone at C-15 with an active amine. Examples of reactive amines include methyl amine and ethyl amine. The products can be reduced or can react with nucleophiles using standard techniques, and the reduction can also extend to reduce the alkenes, if desired, using a reagent such as hydrogen gas over palladium on carbon. Alternatively, sodium cyanoborohydride will selectivity reduce the imine without disrupting the alkenes. Finally, a suitable nucleophile, preferably such as a methyl cerium reagent, can add to the imine. Addition of the methylcerium nucleophile (˜1.5 equiv.) is described in T. Imamoto, et al., “Carbon-Carbon Bond Forming Reactions Using Cerium Metal or Organocerium (III) Reagents”, J. Org. Chem. Vol. 49 (1984) p. 3904-12; T. Imamoto, et al., “Reactions of Carbonyl Compounds with Grignard Reagents in the Presence of Cerium Chloride”, J. Am. Chem. Soc. Vol. 111 (1989) p. 4392-98; and references cited therein, gives the aminomethyl derivative. In that case, R⁵ in compound S1n would be a methyl group.

Using the reactions disclosed above for compounds of the type S2h, compounds of Formula VII can be made from S2n.

### Compositions of the Invention

This invention further relates to a composition for treating hair loss. The composition comprises A) the prostaglandin described above and B) a carrier. The composition may further comprise C) one or more optional activity enhancers.

The composition can be a pharmaceutical or cosmetic composition, administered for treatment or prophylaxis of hair loss. Standard pharmaceutical formulation techniques are used, such as those disclosed in Remington's Pharmaceutical Sciences, Mack Publishing Company, Easton, Pa.. (1990).

The composition further comprises component B) a carrier. “Carrier” means one or more compatible substances that are suitable for administration to a mammal. Carrier includes solid or liquid diluents, hydrotopes, surface-active agents, and encapsulating substances. “Compatible” means that the components of the composition are capable of being commingled with the prostaglandins, and with each other, in a manner such that there is no interaction which would substantially reduce the efficacy of the composition under ordinary use situations. Carriers must be of sufficiently high purity and sufficiently low toxicity to render them suitable for administration to the mammal being treated. The carrier can be inert, or it can possess pharmaceutical benefits, cosmetic benefits, or both.

The choice of carrier for component B) depends on the route by which component A) will be administered and the form of the composition. The composition may be in a variety of forms, suitable, for example, for systemic administration (e.g., oral, rectal, nasal, sublingual, buccal, or parenteral) or topical administration (e.g., local application on the skin, ocular, liposome delivery systems, or iontophoresis). Topical administration directly to the locus of desired hair growth is preferred.

Carriers for systemic administration typically comprise one or more ingredients selected from the group consisting of a) diluents, b) lubricants, c) binders, d) disintegrants, e) colorants, f) flavors, g) sweeteners, h) antioxidants, j) preservatives, k) glidants, m) solvents, n) suspending agents, o) surfactants, combinations thereof, and others.

Ingredient a) is a diluent. Suitable diluents include sugars such as glucose, lactose, dextrose, and sucrose; polyols such as propylene glycol; calcium carbonate; sodium carbonate; glycerin; mannitol; and sorbitol.

Ingredient b) is a lubricant. Suitable lubricants are exemplified by solid lubricants including silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma.

Ingredient c) is a binder. Suitable binders include polyvinylpyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose and sodium carboxymethylcellulose.

Ingredient d) is a disintegrant. Suitable disintegrants include agar, alginic acid and the sodium salt thereof, effervescent mixtures, croscarmelose, crospovidone, sodium carboxymethyl starch, sodium starch glycolate, clays, and ion exchange resins.

Ingredient e) is a colorant such as an FD&C dye.

Ingredient f) is a flavor such as menthol, peppermint, and fruit flavors.

Ingredient g) is a sweetener such as aspartame and saccharin.

Ingredient h) is an antioxidant such as butylated hydroxyanisole, butylated hydroxytoluene, and vitamin E.

Ingredient j) is a preservative such as methyl paraben and sodium benzoate.

Ingredient k) is a glidant such as silicon dioxide.

Ingredient m) is a solvent, such as water, isotonic saline, ethyl oleate, alcohols such as ethanol, and phosphate buffer solutions.

Ingredient n) is a suspending agent. Suitable suspending agents include AVICEL® RC-591 from FMC Corporation of Philadelphia, Pa. and sodium alginate.

Ingredient o) is a surfactant such as the TWEENS® from Atlas Powder Company of Wilmington, Del., lecithin, polysorbate 80, and sodium lauryl sulfate.

Compositions for parenteral administration typically comprise A) 0.1 to 10% of a prostaglandin and B) 90 to 99.9% of a carrier comprising a) a diluent and m) a solvent. Preferably, component a) is propylene glycol and m) is ethanol or ethyl oleate.

Compositions for oral administration can have various dosage forms. For example, solid forms include tablets, capsules, granules, and bulk powders. These oral dosage forms comprise a safe and effective amount, usually at least 5%, and preferably from 25% to 50%, of A) the prostaglandin. The oral dosage compositions further comprise B) 50 to 95% of a carrier, preferably 50 to 75%.

Tablets can be compressed, tablet triturates, enteric-coated, sugar-coated, film-coated, or multiple-compressed. Tablets typically comprise A) the prostaglandin, and B) a carrier comprising ingredients selected from the group consisting of a) diluents, b) lubricants, c) binders. d) disintegrants, e) colorants, f) flavors, g) sweeteners, k) glidants, and combinations thereof. Preferred diluents include calcium carbonate, sodium carbonate, mannitol, lactose and cellulose. Preferred binders include starch, gelatin, and sucrose. Preferred disintegrants include alginic acid, and croscarmelose. Preferred lubricants include magnesium stearate, stearic acid, and talc. Preferred colorants are the FD&C dyes, which can be added for appearance. Chewable tablets preferably contain g) sweeteners such as aspartame and saccharin, or f) flavors such as menthol, peppermint, and fruit flavors.

Capsules (including time release and sustained release formulations) typically comprise A) the prostaglandin, and B) a carrier comprising one or more a) diluents disclosed above in a capsule comprising gelatin. Granules typically comprise A) the prostaglandin, and preferably further comprise k) glidants such as silicon dioxide to improve flow characteristics.

The selection of ingredients in the carrier for oral compositions depends on secondary considerations like taste, cost, and shelf stability, which are not critical for the purposes of this invention. One skilled in the art can optimize appropriate ingredients without undue experimentation.

The solid compositions may also be coated by conventional methods, typically with pH or time-dependent coatings, such that A) the prostaglandin is released in the gastrointestinal tract at various times to extend the desired action. The coatings typically comprise one or more components selected from the group consisting of cellulose acetate phthalate, polyvinylacetate phthalate, hydroxypropyl methyl cellulose phthalate, ethyl cellulose, EUDRAGIT® coatings (available from Rohm & Haas G.M.B.H. of Darmstadt, Germany), waxes and shellac.

Compositions for oral administration can also have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically comprise A) the prostaglandin and B) a carrier comprising ingredients selected from the group consisting of a) diluents, e) colorants, and f) flavors, g) sweeteners, j) preservatives, m) solvents, n) suspending agents, and o) surfactants. Peroral liquid compositions preferably comprise one or more ingredients selected from the group consisting of e) colorants, f) flavors, and g) sweeteners.

Other compositions useful for attaining systemic delivery of the subject compounds include sublingual, buccal and nasal dosage forms. Such compositions typically comprise one or more of soluble filler substances such as a) diluents including sucrose, sorbitol and mannitol; and c) binders such as acacia, microcrystalline cellulose, carboxymethylcellulose, and hydroxypropylmethylcellulose. Such compositions may further comprise b) lubricants, e) colorants, f) flavors, g) sweeteners, h) antioxidants, and k) glidants.

The compositions may further comprise component C) an optional activity enhancer. Component C) is preferably selected from the group consisting of i) hair growth stimulants (other than component A) and ii) penetration enhancers.

Component i) is an optional hair growth stimulant. Component i) is exemplified by vasodilators, antiandrogens, cyclosporins, cyclosporin analogs, antimicrobials, anti-inflammatories, thyroid hormones, thyroid hormone derivatives, and thyroid hormone analogs, non-selective prostaglandin agonists or antagonists, retinoids, triterpenes, combinations thereof, and others. “Non-selective prostaglandin” agonists and antagonists differ from component A) in that they do not selectively activate the FP receptor, and they may activate other receptors.

Vasodilators such as potassium channel agonists including minoxidil and minoxidil derivatives such as aminexil and those described in U.S. Pat. Nos. 3,382,247, 5,756,092, 5,772,990, 5,760,043, 5,466,694, 5,438,058, 4,973,474, and cromakalin and diazoxide can be used as optional hair growth stimulants in the composition.

Examples of suitable antiandrogens include 5-α-reductase inhibitors such as finasteride and those described in U.S. Pat. No. 5,516,779, and in Nane et al., Cancer Research 58, “Effects of Some Novel Inhibitors of CT7,20-Lyase and 5α-Reductase in vitro and in vivo and Their Potential Role in the Treatment of Prostate Cancer,” as well as cyproterone acetate, azelaic acid and its derivatives and those compounds described in U.S. Pat. No. 5,480,913, flutamide, and those compounds described in U.S. Pat. Nos. 5,411,981, 5,565,467, and 4,910,226.

Antimicrobials include selenium sulfide, ketoconazole, triclocarbon, triclosan, zinc pyrithione, itraconazole, asiatic acid, hinokitiol, mipirocin and those described in EPA 0,680,745, clinacycin hydrochloride, benzoyl peroxide, benzyl peroxide and minocyclin.

Examples of suitable anti-inflammatories include glucocorticoids such as hydrocortisone, mometasone furoate and prednisolone, nonsteroidal anti-inflammatories including cyclooxygenase or lipoxygenase inhibitors such as those described in U.S. Pat. No. 5,756,092, and benzydamine, salicylic acid, and those compounds described in EPA 0,770,399, published May 2, 1997, WO 94/06434, published Mar. 31, 1994, and FR 2,268,523, published Nov. 21, 1975.

3,5,3′-Triiodothyronine is an example of a suitable thyroid hormone.

Examples of suitable non-selective prostaglandins agonists and antagonists include compounds such as those described in WO 98/33497, Johnstone, published Aug. 6, 1998, WO 95/11003, Stjemschantz, published Apr. 27, 1995, JP 97-100091, Ueno and JP 96-134242, Nakamura.

Suitable retinoids include isotretinoin, acitretin, and tazarotene.

Other optional hair growth stimulants for component i) include benzalkonium chloride, benzethonium chloride, phenol, estradiol, chlorpheniramine maleate, chlorophyllin derivatives, cholesterol, salicylic acid, cysteine, methionine, red pepper tincture, benzyl nicotinate, D,L-menthol, peppermint oil, calcium pantothenate, panthenol, castor oil, prednisolone, resorcinol, chemical activators of protein kinase C, glycosaminoglycan chain cellular uptake inhibitors, inhibitors of glycosidase activity, glycosaminoglycanase inhibitors, esters of pyroglutamic acid, hexosaccharic acids or acylated hexosaccharic acids, aryl-substituted ethylenes, N-acylated amino acids, flavinoids, ascomycin derivatives and analogs, histamine antagonists such as diphenhydramine hydrochloride, triterpenes such as oleanolic acid and ursolic acid and those described in U.S. Pat. Nos. 5,529,769, 5,468,888, 5,631,282, and 5,679,705, JP 10017431, WO 95/35103, JP 09067253, WO 92/09262, JP 62093215, and JP 08193094; saponins such as those described in EP 0,558,509 to Bonte et al., published Sep. 8, 1993 and WO 97/01346 to Bonte et al, published Jan. 16, 1997, proteoglycanase or glycosaminoglycanase inhibitors such as those described in U.S. Pat. Nos. 5,015,470, 5,300,284, and 5,185,325, estrogen agonists and antagonists, pseudoterins, cytokine and growth factor promoters, analogs or inhibitors such as interleukin 1 inhibitors, interleukin-6 inhibitors, interleukin-10 promoters, and tumor necrosis factor inhibitors, vitamins such as vitamin D analogs and parathyroid hormone antagonists, Vitamin B12 analogs and panthenol, interferon agonists and antagonists, hydroxyacids such as those described in U.S. Pat. No. 5,550,158, benzophenones, and hydantoin anticonvulsants such as phenytoin, and combinations thereof.

Other additional hair growth stimulants are described in JP 09-157,139 to Tsuji et al., published Jun. 17, 1997; EP 0277455 Al to Mirabeau, published Aug. 10, 1988; WO 97/05887 to Cabo Soler et al., published Feb. 20, 1997; WO 92/16186 to Bonte et al., published Mar. 13, 1992; JP 62-93215 to Okazaki et al., published Apr. 28, 1987; U.S. Pat. No. 4,987,150 to Kurono et al., issued Jan. 22, 1991; JP 290811 to Ohba et al., published Oct. 15, 1992; JP 05-286,835 to Tanaka et al., published Nov. 2, 1993, FR 2,723,313 to Greff, published Aug. 2, 1994, U.S. Pat. No. 5,015,470 to Gibson, issued May 14, 1991, U.S. Pat. No. 5,559,092, issued Sep. 24, 1996, U.S. Pat. No. 5,536,751, issued Jul. 16, 1996, U.S. Pat. No. 5,714,515, issued Feb. 3, 1998, EPA 0,319,991, published Jun. 14, 1989, EPA 0,357,630, published Oct. 6, 1988, EPA 0,573,253, published Dec. 8, 1993, JP 61-260010, published Nov. 18, 1986, U.S. Pat. No. 5,772,990, issued Jun. 30, 1998, U.S. Pat. No. 5,053,410, issued Oct. 1, 1991, and U.S. Pat. No. 4,761,401, issued Aug. 2, 1988.

The most preferred activity enhancers are minoxidil and finasteride, most preferably minoxidil.

Component ii) is a penetration enhancer that can be added to all of the compositions for systemic administration. The amount of component ii), when present in the composition, is typically 1 to 5%. Examples of penetration enhancers include 2-methyl propan-2-ol, propan-2-ol, ethyl-2-hydroxypropanoate, hexan-2,5-diol, polyoxyethylene(2) ethyl ether, di(2-hydroxypropyl) ether, pentan-2,4-diol, acetone, polyoxyethylene(2) methyl ether, 2-hydroxypropionic acid, 2-hydroxyoctanoic acid, propan-1-ol, 1,4-dioxane, tetrahydrofuran, butan-1,4-diol, propylene glycol dipelargonate, polyoxypropylene 15 stearyl ether, octyl alcohol, polyoxyethylene ester of oleyl alcohol, oleyl alcohol, lauryl alcohol, dioctyl adipate, dicapryl adipate, di-isopropyl adipate, di-isopropyl sebacate, dibutyl sebacate, diethyl sebacate, dimethyl sebacate, dioctyl sebacate, dibutyl suberate, dioctyl azelate, dibenzyl sebacate, dibutyl phthalate, dibutyl azelate, ethyl myristate, dimethyl azelate, butyl myristate, dibutyl succinate, didecyl phthalate, decyl oleate, ethyl caproate, ethyl salicylate, isopropyl palmitate, ethyl laurate, 2-ethyl-hexyl pelargonate, isopropyl isostearate, butyl laurate, benzyl benzoate, butyl benzoate, hexyl laurate, ethyl caprate, ethyl caprylate, butyl stearate, benzyl salicylate, 2-hydroxypropanoic acid, 2-hydroxyoctanoic acid, dimethyl sulfoxide, N,N-dimethyl acetamide, N,N-dimethyl formamide, 2-pyrrolidone, 1-methyl-2-pyrrolidone, 5-methyl-2-pyrrolidone, 1,5-dimethyl-2-pyrrolidone, 1-ethyl-2-pyrrolidone, phosphine oxides, sugar esters, tetrahydrofurfuryl alcohol, urea, diethyl-m-toluamide, 1-dodecylazacyloheptan-2-one, omega three fatty acids and fish oils, and combinations thereof.

In a preferred embodiment of the invention, the prostaglandins are topically administered. Topical compositions that can be applied locally to the skin may be in any form including solutions, oils, creams, ointments, gels, lotions, shampoos, leave-on and rinse-out hair conditioners, milks, cleansers, moisturizers, sprays, skin patches, and the like. Topical compositions comprise: component A) the prostaglandin described above and component B) a carrier. The carrier of the topical composition preferably aids penetration of the prostaglandins into the skin to reach the environment of the hair follicle. Component B) may further comprise one or more optional components. Topical compositions preferably further comprise C) one or more of the optional activity enhancers described above.

The exact amounts of each component in the topical composition depend on various factors. The amount of component A) added to the topical composition is:

IC₅₀×10⁻²≧% of component A)≧IC₅₀×10⁻³,

where IC₅₀ of component A) is expressed in nanomolar units. “IC₅₀” means inhibitory concentration 50^{th} percentile. For example, if the IC₅₀ of the prostaglandin is 1 nM, the amount of component A) will be 0.001 to 0.01%. If the IC₅₀ of the prostaglandin is 10 nM, the amount of component A) will be 0.01 to 0.1%. If the IC₅₀ of the prostaglandin is 100 nM, the amount of component A) will be 0.1 to 1.0%. If the IC₅₀ of the prostaglandin is 1000 nM, the amount of component A) will be 1.0 to 10%, preferably 1.0 to 5%. If the amount of component A) is outside the ranges specified above (i.e., either higher or lower), efficacy of the treatment may be reduced. IC₅₀ can be calculated according to the method in Reference Example 1, below. One skilled in the art can calculate IC₅₀ without undue experimentation.

The topical composition preferably further comprises 1 to 20% component C), and a sufficient amount of component B) such that the amounts of components A), B), and C), combined equal 100%. The amount of B) the carrier employed in conjunction with component A) is sufficient to provide a practical quantity of composition for administration per unit dose of the prostaglandin. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2^{nd} Ed., (1976).

Component B) the carrier may comprise a single ingredient or a combination of two or more ingredients. In the topical compositions, component B) is a topical carrier. Preferred topical carriers comprise one or more ingredients selected from the group consisting of water, alcohols, aloe vera gel, allantoin, glycerin, vitamin A and E oils, mineral oil, propylene glycol, polypropylene glycol-2 myristyl propionate, dimethyl isosorbide, combinations thereof, and the like. More preferred carriers include propylene glycol, dimethyl isosorbide, and water.

The topical carrier may comprise one or more ingredients selected from the group consisting of q) emollients, r) propellants, s) solvents, t) humectants, u) thickeners, v) powders, and w) fragrances in addition to, or instead of, the preferred topical carrier ingredients listed above. One skilled in the art would be able to optimize carrier ingredients for the topical compositions without undue experimentation.

Ingredient q) is an emollient. The amount of ingredient q) in the topical composition is typically 5 to 95%. Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petrolatum, mineral oil, butyl myristate, isostearic acid, palmitic acid, isopropyl linoleate, lauryl lactate, myristyl lactate, decyl oleate, myristyl myristate, polydimethylsiloxane, and combinations thereof. Preferred emollients include stearyl alcohol and polydimethylsiloxane.

Ingredient r) is a propellant. The amount of ingredient r) in the topical composition is typically 5 to 95%. Suitable propellants include propane, butane, isobutane, dimethyl ether, carbon dioxide, nitrous oxide, and combinations thereof.

Ingredient s) is a solvent. The amount of ingredient s) in the topical composition is typically 5 to 95%. Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Preferred solvents include ethyl alcohol.

Ingredient t) is a humectant. The amount of ingredient t) in the topical composition is typically 5 to 95%. Suitable humectants include glycerin, sorbitol, sodium 2-pyrrolidone-5-carboxylate, soluble collagen, dibutyl phthalate, gelatin, and combinations thereof. Preferred humectants include glycerin.

Ingredient u) is a thickener. The amount of ingredient u) in the topical composition is typically 0 to 95%.

Ingredient v) is a powder. The amount of ingredient v) in the topical composition is typically 0 to 95%. Suitable powders include chalk, talc, fullers earth, kaolin, starch, gums, colloidal silicon dioxide, sodium polyacrylate, tetra alkyl ammonium smectites, trialkyl aryl ammonium smectites, chemically modified magnesium aluminum silicate, organically modified montmorillonite clay, hydrated aluminum silicate, fumed silica, carboxyvinyl polymer, sodium carboxymethyl cellulose, ethylene glycol monostearate, and combinations thereof.

Ingredient w) is a fragrance. The amount of ingredient w) in the topical composition is typically 0.001 to 0.5%, preferably 0.001 to 0.1%.

Component C) the optional activity enhancer is as described above. Any of the i) hair growth stimulants and ii) penetration enhancers may be added to the topical compositions. Preferably, the topical composition comprises 0.01 to 15% of component i) the optional hair growth stimulant. More preferably, the composition comprises 0.1 to 10%, and most preferably 0.5 to 5% of component i). Preferably, the topical composition comprises 1 to 5% of component ii).

In an alternative embodiment of the invention, topical pharmaceutical compositions for ocular administration are prepared by conventional methods. Topical pharmaceutical compositions for ocular administration typically comprise A) a prostaglandin B) a carrier, such as purified water, and one or more ingredients selected from the group consisting of y) sugars such as dextrans, particularly dextran 70, z) cellulose or a derivative thereof, aa) a salt, bb) disodium EDTA (Edetate disodium), and cc) a pH adjusting additive.

Examples of z) cellulose derivatives suitable for use in the topical pharmaceutical composition for ocular administration include sodium carboxymethylcellulose, ethylcellulose, methylcellulose, and hydroxypropylmethylcellulose. Hydroxypropylmethylcellulose is preferred.

Examples of aa) salts suitable for use in the for use in the topical pharmaceutical composition for ocular administration include sodium chloride, potassium chloride, and combinations thereof.

Examples of cc) pH adjusting additives include HCl or NaOH in amounts sufficient to adjust the pH of the topical pharmaceutical composition for ocular administration to 7.2-7.5.

This invention further relates to a method for darkening hair, thickening hair, and reversing hair graying. The method comprises applying the topical composition for treating hair loss to hair, to skin in the locus of hair, or both. For example, the topical composition may be applied to hair growing on the scalp or eyelashes. The topical composition can be, for example, a cosmetic composition prepared as described above. An example of a composition that may be applied to eyelashes is a mascara. The prostaglandin may be added to mascara compositions known in the art, such as the mascara described in U.S. Pat. No. 5,874,072, which is hereby incorporated by reference. The mascara comprises dd) a water-insoluble material, ee) a water-soluble, film-forming polymer, ff) a wax, o) a surfactant, gg) a pigment, and s) a solvent.

Ingredient dd) is a water-insoluble material selected from the group consisting of acrylate copolymers; styrene/acrylate/methacrylate copolymers; acrylic latex; styrene/acrylic ester copolymer latex; polyvinylacetate latex; vinyl acetate/ethylene copolymer latex; styrene/butadiene copolymer latex; polyurethane latex; butadiene/acrylonitrile copolymer latex; styrene/acrylate/acrylonitrile copolymer latex; and mixtures thereof, wherein the acrylate copolymers, and the styrene/acrylate/methacrylate copolymers additionally comprise ammonia, propylene glycol, a preservative and a surfactant.

Ingredient ee) is a water-soluble, film-forming polymer. Ingredient ee) is selected from the group consisting of vinyl alcohol/poly(alkyleneoxy)acrylate, vinyl alcohol/vinyl acetate/poly-(alkyleneoxy)acrylate, polyethylene oxide, polypropylene oxide, acrylates/octyl-acrylamide copolymers and mixtures thereof.

Ingredient ff) is a wax. “Wax” means a lower-melting organic mixture or compound of high molecular weight, solid at room temperature and generally similar in composition to fats and oils except that they contain no glycerides. Some are hydrocarbons, others are esters of fatty acids and alcohols. Waxes useful in this invention are selected from the group consisting of animal waxes, vegetable waxes, mineral waxes, various fractions of natural waxes, synthetic waxes, petroleum waxes, ethylenic polymers, hydrocarbon types such as Fischer-Tropsch waxes, silicone waxes, and mixtures thereof wherein the waxes have a melting point between 55 and 100° C.

Ingredient o) is surfactant, as described above. Ingredient o) in the mascara is preferably a surfactant having an HLB from 3 to 15. Suitable surfactants include those disclosed in the C.T.F.A. Cosmetic Ingredient Handbook, pp. 587-592 (1992); Remington's Pharmaceutical Sciences, 15th ed., pp. 335-337 (1975); and McCutcheon's Volume 1, Emulsifiers & Detergents, North American Edition, pp. 236-239 (1994).

Ingredient gg) is a pigment. Suitable pigments include inorganic pigments, organic lake pigments, pearlescent pigments, and mixtures thereof. Inorganic pigments useful in this invention include those selected from the group consisting of rutile or anatase titanium dioxide, coded in the Color Index under the reference CI 77,891; black, yellow, red and brown iron oxides, coded under references CI 77,499, 77,492 and, 77,491; manganese violet (CI 77,742); ultramarine blue (CI 77,007); chromium oxide (CI 77,288); chromium hydrate (CI 77,289); and ferric blue (CI 77,510); and mixtures thereof.

The organic pigments and lakes useful in this invention include those selected from the group consisting of D&C Red No. 19 (CI 45,170), D&C Red No. 9 (CI 15,585), D&C Red No. 21 (CI 45,380), D&C Orange No. 4 (CI 15,510), D&C Orange No. 5 (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430), and the dye or lakes based on Cochineal Carmine (CI 75,570), and mixtures thereof.

The pearlescent pigments useful in this invention include those selected from the group consisting of the white pearlescent pigments such as mica coated with titanium oxide, bismuth oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof.

Ingredient s) is a solvent described above, preferably water.

The amount of A) the prostaglandin added to the mascara is as described above for topical compositions.

The prostaglandins may also be administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines. A preferred formulation for topical delivery of the present compounds uses liposomes as described in Dowton et al., “Influence of Liposomal Composition on Topical Delivery of Encapsulated Cyclosporin A: I. An in vitro Study Using Hairless Mouse Skin”, S.T.P. Pharma Sciences, Vol. 3, pp. 404-407 (1993); Wallach and Philippot, “New Type of Lipid Vesicle: Novasome®”, Liposome Technology, Vol. 1, pp. 141-156 (1993); Wallach, U.S. Pat. No. 4,911,928, assigned to Micro-Pak, Inc., issued Mar. 27, 1990; and Weiner et al., U.S. Pat. No. 5,834,014, assigned to The University of Michigan and Micro-Pak, Inc., issued Nov. 10, 1998 (with respect to Weiner et al., with a compound as described herein administered in lieu of, or in addition to, minoxidil).

The prostaglandins may also be administered by iontophoresis. See, e.g., Internet site www.unipr.it/arpa/dipfarm/erasmus/erasm14.html; Banga et al., “Hydrogel-based Iontotherapeutic Delivery Devices for Transdermal Delivery of Peptide/Protein Drugs”, Pharm. Res., Vol. 10 (5), pp. 697-702 (1993); Ferry, “Theoretical Model of Iontophoresis Utilized in Transdermal Drug Delivery”, Pharmaceutical Acta Helvetiae, Vol. 70, pp. 279-287 (1995); Gangarosa et al., “Modern Iontophoresis for Local Drug Delivery”, Int. J. Pharm., Vol. 123, pp. 159-171 (1995); Green et al., “Iontophoretic Delivery of a Series of Tripeptides Across the Skin in vitro”, Pharm. Res., Vol 8, pp. 1121-11²⁷ (1991); Jadoul et al., “Quantification and Localization of Fentanyl and TRH Delivered by Iontophoresis in the Skin”, Int. J. Pharm., Vol. 120, pp. 221-8 (1995); O'Brien et al., “An Updated Review of its Antiviral Activity, Pharmacokinetic Properties and Therapeutic Efficacy”, Drugs, Vol. 37, pp. 233-309 (1989); Parry et al., “Acyclovir Biovailability in Human Skin”, J. Invest. Dermatol., Vol. 98 (6), pp. 856-63 (1992); Santi et al., “Drug Reservoir Composition and Transport of Salmon Calcitonin in Transdermal Iontophoresis”, Pharm. Res., Vol 14 (1), pp. 63-66 (1997); Santi et al., “Reverse Iontophoresis—Parameters Determining Electroosmotic Flow: I. pH and Ionic Strength”, J. Control. Release, Vol. 38, pp. 159-165 (1996); Santi et al., “Reverse Iontophoresis—Parameters Determining Electroosmotic Flow: II. Electrode Chamber Formulation”, J. Control. Release, Vol. 42, pp. 29-36 (1996); Rao et al., “Reverse Iontophoresis: Noninvasive Glucose Monitoring in vivo in Humans”, Pharm. Res., Vol. 12 (12), pp. 1869-1873 (1995); Thysman et al., “Human Calcitonin Delivery in Rats by Iontophoresis”, J. Pharm. Pharmacol., Vol. 46, pp. 725-730 (1994); and Volpato et al., “Iontophoresis Enhances the Transport of Acyclovir through Nude Mouse Skin by Electrorepulsion and Electroosmosis”, Pharm. Res., Vol. 12 (11), pp. 1623-1627 (1995).

The prostaglandins may be included in kits comprising a prostaglandin, a systemic or topical composition described above, or both; and information, instructions, or both that use of the kit will provide treatment for hair loss in mammals (particularly humans). The information and instructions may be in the form of words, pictures, or both, and the like. In addition or in the alternative, the kit may comprise a prostaglandin, a composition, or both; and information, instructions, or both, regarding methods of application of the prostaglandin or composition, preferably with the benefit of treating hair loss in mammals.

### Methods of the Invention

This invention further relates to a method for treating hair loss in mammals. The method comprises administering to a mammal (preferably a human) suffering from hair loss, a prostaglandin described above. For example, a mammal diagnosed with alopecia including male pattern baldness and female pattern baldness can be treated by the methods of this invention. Preferably, a systemic or topical composition comprising A) the prostaglandin and B) a carrier is administered to the mammal. More preferably, the composition is a topical composition comprising A) the prostaglandin, B) the carrier, and C) an optional activity enhancer.

The dosage of the prostaglandin administered depends on the method of administration. For systemic administration, (e.g., oral, rectal, nasal, sublingual, buccal, or parenteral), typically, 0.5 mg to 300 mg, preferably 0.5 mg to 100 mg, more preferably 0.1 mg to 10 mg, of a prostaglandin described above is administered per day. These dosage ranges are merely exemplary, and daily administration can be adjusted depending on various factors. The specific dosage of the prostaglandin to be administered, as well as the duration of treatment, and whether the treatment is topical or systemic are interdependent. The dosage and treatment regimen will also depend upon such factors as the specific prostaglandin used, the treatment indication, the efficacy of the compound, the personal attributes of the subject (such as, for example, weight, age, sex, and medical condition of the subject), compliance with the treatment regimen, and the presence and severity of any side effects of the treatment.

For topical administration (e.g., local application on the skin, ocular, liposome delivery systems, or iontophoresis), the topical composition is typically administered once per day. The topical compositions are administered daily for a relatively short amount of time (i.e., on the order of weeks). Generally, 6 to 12 weeks is sufficient. The topical compositions are preferably leave-on compositions. In general, the topical composition should not be removed for at least several hours after administration.

In addition to the benefits in treating hair loss, the prostaglandins in the compositions and methods of this invention also darken and thicken hair and may reverse hair graying. This invention further relates to a method for darkening and thickening hair. The method comprises applying the topical composition for treating hair loss to growing hair. In a preferred embodiment of the invention, the topical composition, such as the mascara composition described above, is applied to eyelashes.

### EXAMPLES

These examples are intended to illustrate the invention to those skilled in the art and should not be interpreted as limiting the scope of the invention set forth in the claims.

### Reference Example 1

Radioligand Binding Assay

IC₅₀ of a prostaglandin can be determined relative to PGF_{2α} using the Radioligand Binding Assay. As a control, the IC₅₀ for PGF_{2α} itself should be no lower than 1.0 nM and no higher than 5.0 nM.

In this assay, COS-7 cells are transiently transfected with the hFP recombinant plasmid using LipofectAMINE Reagent. Forty-eight hours later, the tranfected cells are washed with Hank's Balanced Salt Solution (HBSS, without CaCl₂, MgCl₂, MgSO₄, or phenol red). The cells are detached with versene, and HBSS is added. The mixture is centrifuged at 200 g for 10 minutes, at 4° C. to pellet the cells. The pellet is resuspended in Phosphate-Buffered Saline-EDTA buffer (PBS; 1 mM EDTA; pH 7.4; 4° C). The cells are disrupted by nitrogen cavitation (Parr model 4639), at 800 psi, for 15 minutes at 4° C. The mixture is centrifuged at 1000 g for 10 minutes at 4° C. The supernatant is centrifuged at 100,000 g for 60 minutes at 4° C. The pellet is resuspended to 1 mg protein/mL TME buffer (50 mM Tris; 10 mM MgCl2; 1 mM EDTA; pH 6.0; 4° C.) based on protein levels measured using the Pierce BCA Protein Assay kit. The homogenate is mixed for 10 seconds using a Kinematica POLYTRON® (available from KINEMATICA AG, Luzernerstrasse147A CH-6014 Littau, Switzerland). The membrane preparations are then stored at −80° C., until thawed for assay use.

The receptor competition binding assays are developed in a 96 well format. Each well contains 100 g of hFP membrane, 5 nM (3 H) PGF2, and the various competing compounds in a total volume of 200 L. The plates are incubated at 23° C. for 1 hour. The incubation is terminated by rapid filtration using the Packard Filtermate 196 harvester through Packard UNIFILTER® GF/B filters (available from Packard Instrument Co., Inc. of Downers Grove Ill.) pre-wetted with TME buffer. The filter is washed four times with TME buffer. Packard Microscint 20, a high efficiency liquid scintillation cocktail, is added to the filter plate wells and the plates remain at room temperature for three hours prior to counting. The plates are read on a Packard TOPCOUNT® Microplate Scintillation Counter (also available from Packard Instrument Co., Inc.)

### Reference Example 2

Telogen Conversion Assay

Prostaglandins are tested for their potential to grow hair using the Telogen Conversion Assay. The Telogen Conversion Assay measures the potential of a prostaglandin to convert mice in the resting stage of the hair growth cycle (“telogen”), to the growth stage of the hair growth cycle (“anagen”).

Without intending to be limited by theory, there are three principal phases of the hair growth cycle: anagen, catagen, and telogen. It is believed that there is a longer telogen period in C3H mice (Harlan Sprague Dawley, Inc., Indianapolis, Ind.) from approximately 40 days of age until about 75 days of age, when hair growth is synchronized. It is believed that after 75 days of age, hair growth is no longer synchronized. Wherein about 40 day-old mice with dark fur (brown or black) are used in hair growth experiments, melanogenesis occurs along with hair (fur) growth wherein the topical application of hair growth inducers are evaluated. The Telogen Conversion Assay herein is used to screen prostaglandins for potential hair growth by measuring melanogenesis.

Three groups of 44 day-old C3H mice are used: a vehicle control group, a positive control group, and a test prostaglandin group, wherein the test prostaglandin group is administered a prostaglandin used in the method of this invention. The length of the assay is 24 days with 15 treatment days (wherein the treatment days occur Mondays through Fridays). Day 1 is the first day of treatment. A typical study design is shown in Table 3 below. Typical dosage concentrations are set forth in Table 3, however the skilled artisan will readily understand that such concentrations may be modified.

TABLE 3
Assay Parameters

| | | | | | Length |
|---|---|---|---|---|---|
| Group | Animal | | | Application | of |
| # | # | Compound | Concentration | volume | Study |
| 1 | 1-10 | Test | 0.01% in | 400 μL | 26 days |
| | | Compound | vehicle** | topical | |
| 2 | 11-20 | Positive | 0.01% in | 400 μL | 26 days |
| | | Control | vehicle** | topical | |
| | | (T3)* | | | |
| 3 | 21-30 | Vehicle** | N/A | 400 μL | 26 days |
| | | | | topical | |

*T3 is 3,5,3′-triiodothyronine.
**The vehicle is 60% ethanol, 20% propylene glycol, and 20% dimethyl isosorbide (commercially available from Sigma Chemical Co., St. Louis, MO).

The mice are treated topically Monday through Friday on their lower back (base of tail to the lower rib). A pipettor and tip are used to deliver 400 μL to each mouse's back. The 400 μL application is applied slowly while moving hair on the mouse to allow the application to reach the skin.

While each treatment is being applied to the mouse topically, a visual grade of from 0 to 4 will be given to the skin color in the application area of each animal. As a mouse converts from telogen to anagen, its skin color will become more bluish-black. As indicated in Table 4, the grades 0 to 4 represent the following visual observations as the skin progresses from white to bluish-black.

TABLE 4
Evaluation Criteria

| Visual Observation | Grade |
|---|---|
| Whitish Skin Color | 0 |
| Skin is light gray (indication of initiation of anagen) | 1 |
| Appearance of Blue Spots | 2 |
| Blue Spots are aggregating to form one large blue area | 3 |
| Skin is dark blue (almost black) with color covering majority of | 4 |
| treatment area (indication of mouse in full anagen) | |

### Example 1

Compositions for topical administration are made, comprising:

| Component | 1-1 | 1-2 |
|---|---|---|
| Prostaglandin (wt %) | 0.01 | 0.1 |
| IC₅₀ of the Prostaglandin (nM) | 15 | 150 |
| Ethanol (wt %) | 59.99 | 59.9 |
| Propylene Glycol (wt %) | 20.00 | 20.0 |
| Dimethyl Isosorbide (wt %) | 20.00 | 20.0 |

The prostaglandins used are shown below:

| Sample | Prostaglandin |
|---|---|
| 1-1 | 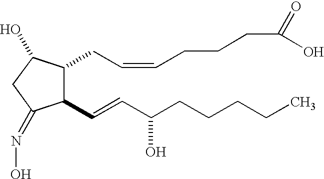 |
| 1-2 | 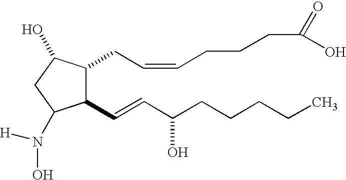 |

A human male subject suffering from male pattern baldness is treated by a method of this invention. Specifically, for 6 weeks, one of the above compositions is daily administered topically to the subject to induce hair growth.

### Example 2

A composition for topical administration is made according to the method of Dowton et al., “Influence of Liposomal Composition on Topical Delivery of Encapsulated Cyclosporin A: I. An in vitro Study Using Hairless Mouse Skin”, S.T.P. Pharma Sciences, Vol. 3, pp. 404-407 (1993), using a PGF in lieu of cyclosporin A and using the NOVASOME® 1 (available from Micro-Pak, Inc. of Wilmington, Del.) for the non-ionic liposomal formulation.

A human male subject suffering from male pattern baldness is treated each day with the above composition. Specifically, for 6 weeks, the above composition is administered topically to the subject.

### Example 3

| Component | Ex. 3-1 | Ex. 3-2 | Ex. 3-3 | Ex. 3-4 |
|---|---|---|---|---|
| Ammonium Lauryl Sulfate | 11.5% | 11.5% | 9.5% | 7.5% |
| Ammonium Laureth Sulfate | 4% | 3% | 2% | 2% |
| Cocamide MEA | 2% | 2% | 2% | 2% |
| Ethylene Glycol Distearate | 2% | 2% | 2% | 2% |
| Cetyl Alcohol | 2% | 2% | 2% | 2% |
| Stearyl Alcohol | 1.2% | 1.2% | 1.2% | 1.2% |
| Glycerin | 1% | 1% | 1% | 1% |
| Polyquaternium 10 | 0.5% | 0.25% | — | — |
| Polyquaternium 24 | — | — | 0.5% | 0.25% |
| Sodium Chloride | 0.1% | 0.1% | 0.1% | 0.1% |
| Sucrose Polyesters of | 3% | 3% | — | — |
| Cottonate Fatty Acid | | | | |
| Sucrose Polyesters of | 2% | 3% | — | — |
| Behenate Fatty Acid | | | | |
| Polydimethylsiloxane | — | — | 3% | 2% |
| Cocaminopropyl Betaine | — | 1% | 3% | 3% |
| Lauryl Dimethyl | 1.5% | 1.5% | 1.5% | 1.5% |
| Amine Oxide | | | | |
| Decyl Polyglucose | — | — | 1% | 1% |
| DMDM Hydantoin | 0.15% | 0.15% | 0.15% | 0.15% |
| Prostaglandin having IC₅₀ | — | 0.162% | 0.162% | — |
| of 162 nM | | | | |
| Prostaglandin having IC₅₀ | 0.15% | — | — | 0.15% |
| of 150 nM | | | | |
| Minoxidil | | | 3% | 2% |
| Phenoxyethanol | 0.5% | 0.5% | 0.5% | 0.5% |
| Fragrance | 0.5% | 0.5% | 0.5% | 0.5% |
| Water | q.s | q.s | q.s | q.s |

The prostaglandin having IC₅₀ of 162 nM is:

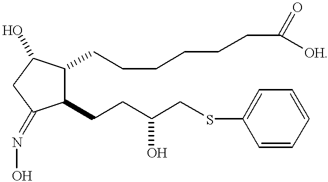

The prostaglandin having IC₅₀ of 150 nM is the same as as that in Example 1-2.

A human subject suffering from male pattern baldness is treated by a method of this invention. Specifically, for 12 weeks, a shampoo described above is used daily by the subject.

### Example 4

A mascara composition is prepared. The composition comprises:

| Component | % W/W |
|---|---|
| WATER, DEIONIZED, USP | q.s. |
| BLACK 1080 MICRONIZED TYPE | 10.000 |
| GLYCERYL MONOSTEARATE (2400 TYPE) | 8.500 |
| C18-36 ACID TRIGLYCERIDE | 5.500 |
| STEARIC ACID, TRIPLE PRESSED, LIQUID | 4.000 |
| ETHYL ALCOHOL SD 40-B, 190 PROOF/SERIAL #: | 4.000 |
| BEESWAX WHITE, FLAKES | 3.250 |
| SHELLAC, NF | 3.000 |
| LECITHIN, GRANULAR (TYPE 6450) | 2.500 |
| TRIETHANOLAMINE 99% - TANK | 2.470 |
| PARAFFIN WAX | 2.250 |
| PARAFFIN WAX 118/125 | 2.250 |
| CARNAUBA WAX, NF | 2.000 |
| POTASSIUM CETYL PHOSPHATE | 1.000 |
| PHENOXYETHANOL | 0.800 |
| OLEIC ACID NF | 0.750 |
| DL-PANTHENOL | 0.350 |
| PVP/VA COPOLYMER | 0.250 |
| METHYLPARABEN, NF | 0.200 |
| DIAZOLIDINYL UREA | 0.200 |
| SIMETHICONE | 0.200 |
| ETHYLPARABEN NF | 0.150 |
| PENTAERYTHRITYL HYDROGENATED ROSINATE | 0.150 |
| PROPYLPARABEN, NF | 0.100 |
| TRISODIUM EDTA | 0.100 |
| PROSTAGLANDIN having IC₅₀ of 15 nM | 0.001 |

The prostaglandin having IC₅₀ of 15 nM is the same as that used in Example 1-1. A human female subject applies the composition each day. Specifically, for 6 weeks, the above composition is administered topically to the subject to darken and thicken eyelashes.

### Example 5

Pharmaceutical compositions in the form of tablets are prepared by conventional methods, such as mixing and direct compaction, formulated as follows:

| | Quantity |
|---|---|
| Ingredient | (mg per tablet) |
| Prostaglandin | 0.5 |
| Microcrystalline Cellulose | 100 |
| Sodium Starch Glycollate | 30 |
| Magnesium Stearate | 3 |

The prostaglandin is the same as that used in Example 3-2

The above composition is administered orally to a subject once daily for 6 to 12 weeks to promote hair growth.

### Example 6

Pharmaceutical compositions in liquid form are prepared by conventional methods, formulated as follows:

| Ingredient | Quantity | |
|---|---|---|
| Prostaglandin | 0.1 | mg |
| Phosphate buffered physiological saline | 10 | ml |
| Methyl Paraben | 0.05 | ml |

The prostaglandin is the same as that used in Example 3-2.

1.0 ml of the above composition is administered subcutaneously at the site of hair loss once daily for 6 to 12 weeks to promote hair growth.

### Example 7

A topical pharmaceutical composition for lowering intraocular pressure is prepared by conventional methods and formulated as follows:

| Ingredient | Amount (wt %) |
|---|---|
| Prostaglandin | 0.004 |
| Dextran 70 | 0.1 |
| Hydroxypropyl methylcellulose | 0.3 |
| Sodium Chloride | 0.77 |
| Potassium chloride | 0.12 |
| Disodium EDTA (Edetate disodium) | 0.05 |
| Benzalkonium chloride | 0.01 |
| HCL and/or NaOH | pH 7.2-7.5 |
| Purified water | q.s. to 100% |

The prostaglandin is the same as that used in Example 3-2.

The above composition is administered ocularly to a subject once per day for 6 to 12 weeks to promote eyelash growth.

### Effects of the Invention

The compositions and methods herein provide a cosmetic benefit with respect to hair growth and appearance in subjects desiring such treatment.

What is claimed is:

1. A composition for treating hair loss comprising:
A) an active ingredient selected from the group consisting of oximyl- and hydroxylamino-prostaglandins having the functionality 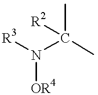
wherein C is a carbon atom bonded within a cyclopentyl ring and wherein the active ingredient selectively activates FP receptors and does not activate any other receptors that negate effects caused by activating the FP receptors, and wherein
R² is hydrogen, and R³ is selected from the group consisting of hydrogen and a lower monovalent hydrocarbon group, with the proviso that alternatively, R² and R³ may form a covalent bond, and
R⁴ is selected from the group consisting of a hydrogen atom, a monovalent hydrocarbon group, a heterogeneous group, a carbocyclic group, heterocyclic group, an aromatic group, a heteroaromatic group, a substituted monovalent hydrocarbon group, a substituted heterogeneous group, a substituted carbocyclic group, a substituted heterocyclic group, a substituted aromatic group, and a substituted heteroaromatic group; and
B) a carrier.

2. The composition of claim 1, wherein R⁴ is selected from the group consisting of a hydrogen atom and a monovalent hydrocarbon group of 1 to 8 carbon atoms.

3. The composition of claim 1, wherein
A) the active ingredient has the structure: 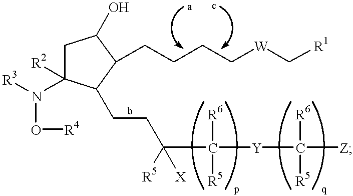
pharmaceutically acceptable salts and hydrates of the structure above; biohydrolyzable amides, esters, and imides of the structure above; and optical isomers, diastereomers, and enantiomers of the structure above; and combinations thereof;
wherein W is selected from the group consisting of an oxygen atom, a sulfur atom, NH, S(O), S(O)₂, and —(CH₂)ₘ—, wherein m is 0 to 3;
X is selected from the group consisting of NHR⁸, OR⁸, SR⁹, and S(O)R⁹;
Y is selected from the group consisting of a bond, an oxygen atom, a sulfur atom, NHR⁸, S(O), and S(O)₂; with the proviso that when Y is NHR⁸, no carbon atom in R⁸ is bonded to more than one heteroatom;
Z is selected from the group consisting of H, CH₃, a carbocyclic group, a heterocyclic group, a substituted carbocyclic group, a substituted heterocyclic group, an aromatic group, a heteroaromatic group, a substituted aromatic group, and a substituted heteroaromatic group;
R¹ is selected from the group consisting of CO₂H, CO₂R⁷, C(O)NHOH, S(O)₂R⁷, C(O)NHS(O)₂R⁷, and tetrazole;
each R⁵ is independently selected from the group consisting of H, CH₃, and C₂H₅;
each R⁶ is independently selected from the group consisting of H, CH₃, C₂H₅, OR⁸, and NHR⁸;
R⁷ is selected from the group consisting of monovalent hydrocarbon groups, heterogeneous groups, aromatic groups, heteroaromatic groups, monocyclic carbocyclic groups, monocyclic heterocyclic groups, substituted monovalent hydrocarbon groups, substituted aromatic groups, and substituted heteroaromatic groups;
each R⁸ is independently selected from the group consisting of a hydrogen atom, an acyl group, a monovalent hydrocarbon group, a substituted monovalent hydrocarbon group, a heterogeneous group, a substituted heterogeneous group, a carbocyclic group, a substituted carbocyclic group, and heterocyclic group, a substituted heterocyclic group, an aromatic group, a substituted aromatic group, a heteroaromatic group, and a substituted heteroaromatic group;
each R⁹ is independently selected from the group consisting of a monovalent hydrocarbon group, a substituted monovalent hydrocarbon group, a heterogeneous group, a substituted heterogeneous group, a carbocyclic group, a substituted carbocyclic group, and heterocyclic group, a substituted heterocyclic group, an aromatic group, a substituted aromatic group, a heteroaromatic group, and a substituted heteroaromatic group;
p is an integer with a value of 0 to 6, q is an integer with a value of 0 to 5, with the proviso that (p+q)=1 to 5, and
bonds a, b, and c are each independently selected from the group consisting of a single bond, a cis double bond, and a trans double bond.

4. The composition of claim 3, wherein W is selected from the group consisting of an oxygen atom and —(CH₂)ₘ—.

5. The composition of claim 3, wherein X is OR⁸.

6. The composition of claim 3, wherein Y is selected from the group consisting of a bond, an oxygen atom, and NHR⁸.

7. The composition of claim 3, wherein Z is selected from the group consisting of aromatic, heteroaromatic, substituted aromatic, and substituted heteroaromatic groups.

8. The composition of claim 3, wherein R¹ is selected from the group consisting of CO₂H, C(O)NHOH, CO₂R⁷, C(O)NHS(O)₂R⁷, and tetrazole.

9. The composition of claim 3, wherein each R⁵ is independently selected from the group consisting of H and CH₃.

10. The composition of claim 3, wherein each R⁶ is independently selected from the group consisting of H, CH₃, C₂H₅, and OR⁸.

11. The composition of claim 3, wherein R⁷ is selected from the group consisting of methyl, ethyl, and isopropyl groups.

12. The composition of claim 3, wherein each R⁸ is a hydrogen atom.

13. The composition of claim 3, wherein p is an integer with a value 1 to 5.

14. The composition of claim 3, wherein bond a is selected from the group consisting of a single bond and a cis double bond.

15. The composition of claim 3, wherein bond b is selected from the group consisting of a single bond and a trans double bond.

16. The composition of claim 3, wherein Y is a bond, p is 0, and q is 2 or 3.

17. The composition of claim 3, wherein Y is a bond, q is 0, and component A) has the structure: 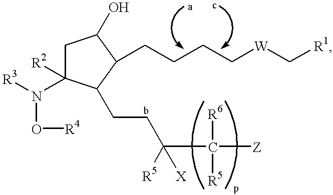
wherein R¹, W, R², R³, R⁴, R⁵, X, R⁶, Z, p, and bonds a, b, and c are as described above.

18. The composition of claim 3, wherein component A) is added in an amount of
IC₅₀×10⁻²≧% of component A)≧IC₅₀×10⁻³,
where IC₅₀ of component A) is expressed in nanomolar units.

19. The composition of claim 18, wherein component C) an activity enhancer is added to the composition in an amount of 1 to 20%, and a sufficient amount of component B) is added such that the amounts of components A), B), and C) combined equal 100%.

20. The composition of claim 3, wherein component B) comprises an ingredient selected from the group consisting of q) emollients, r) propellants, s) solvents, t) humectants, u) thickeners, v) powders, w) fragrances, water, alcohols, aloe vera gel, allantoin, glycerin, vitamin A and E oils, mineral oil, propylene glycol, polypropylene glycol-2 myristyl propionate, dimethyl isosorbide, and combinations thereof.

21. The composition of claim 20, wherein ingredient q) is selected from the group consisting of stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petrolatum, mineral oil, butyl myristate, isostearic acid, palmitic acid, isopropyl linoleate, lauryl lactate, myristyl lactate, decyl oleate, myristyl myristate, polydimethylsiloxane, and combinations thereof.

22. The composition of claim 20, wherein ingredient r) is selected from the group consisting of propane, butane, isobutane, dimethyl ether, carbon dioxide, nitrous oxide, and combinations thereof.

23. The composition of claim 20, wherein ingredient s) is selected from the group consisting of water, ethyl alcohol, methylene chloride, isopropanol, castor oil, ethylene glycol monoethyl ether, diethylene glycol monobutyl ether, diethylene glycol monoethyl ether, dimethyl sulfoxide, dimethyl formamide, tetrahydrofuran, and combinations thereof.

24. The composition of claim 20, wherein ingredient t) is selected from the group consisting of glycerin, sorbitol, sodium 2-pyrrolidone-5-carboxylate, soluble collagen, dibutyl phthalate, gelatin, and combinations thereof.

25. The composition of claim 20, wherein ingredient v) is selected from the group consisting of chalk, talc, fullers earth, kaolin, starch, gums, colloidal silicon dioxide, sodium polyacrylate, tetra alkyl ammonium smectites, trialkyl aryl ammonium smectites, chemically modified magnesium aluminum silicate, organically modified montmorillonite clay, hydrated aluminum s ilicate, fumed silica, carboxyvinyl polymer, sodium carboxymethyl cellulose, ethylene glycol monostearate, and combinations thereof.

26. A method of treating hair loss comprising administering to a mammal a composition comprising:
A) an active ingredient selected from the group consisting of oximyl- and hydroxylamino-prostaglandins having the functionality 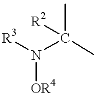
wherein C is a carbon atom bonded within a cyclopentyl ring and wherein the active ingredient selectively activates FP receptors and does not activate any other receptors that negate effects caused by activating the FP receptors, and wherein
R² is hydrogen, and R³ is selected from the group consisting of hydrogen and a lower monovalent hydrocarbon group, with the proviso that alternatively, R² and R³ may form a covalent bond, and
R⁴ is selected from the group consisting of a hydrogen atom, a monovalent hydrocarbon group, a heterogeneous group, a carbocyclic group, heterocyclic group, an aromatic group, a heteroaromatic group, a substituted monovalent hydrocarbon group, a substituted heterogeneous group, a substituted carbocyclic group, a substituted heterocyclic group, a substituted aromatic group, and a substituted heteroaromatic group.

27. The method of claim 26, wherein component A) is selected from the group consisting of oximyl- and hydroxylamino-prostaglandins having the structure: 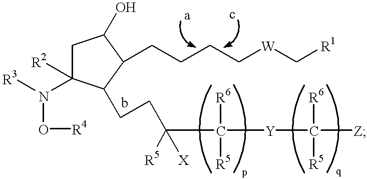
pharmaceutically acceptable salts and hydrates of the structure above; biohydrolyzable amides, esters, and imides of the structure above; optical isomers, diastereomers, and enantiomers of the structure above; and combinations thereof;
wherein W is selected from the group consisting of an oxygen atom, a sulfur atom, NH, S(O), S(O)₂, and —(CH₂)ₘ—, wherein m is 0 to 3;
X is selected from the group consisting of NHR⁸, OR⁸, SR⁹, and S(O)R⁹;
Y is selected from the group consisting of a bond, an oxygen atom, a sulfur atom, NHR⁸, S(O), and S(O)₂; with the proviso that when Y is NHR⁸, no carbon atom in R⁸ is bonded to more than one heteroatom;
Z is selected from the group consisting of H, CH₃, a carbocyclic group, a heterocyclic group, a substituted carbocyclic group, a substituted heterocyclic group, an aromatic group, a heteroaromatic group, a substituted aromatic group, and a substituted heteroaromatic group;
R¹ is selected from the group consisting of CO₂H, C(O)NHOH, S(O)₂R⁷, C(O)NHS(O)₂R⁷, and tetrazole;
each R⁵ is independently selected from the group consisting of H, CH₃, and C₂H₅;
each R⁶ is independently selected from the group consisting of H, CH₃, C₂H₅, OR⁸, and NHR⁸;
R⁷ is selected from the group consisting of monovalent hydrocarbon groups, heterogeneous groups, aromatic groups, heteroaromatic groups, monocyclic carbocyclic groups, monocyclic heterocyclic groups, substituted monovalent hydrocarbon groups, substituted aromatic groups, and substituted heteroaromatic groups;
each R⁸ is independently selected from the group consisting of a hydrogen atom, an acyl group, a monovalent hydrocarbon group, a substituted monovalent hydrocarbon group, a heterogeneous group, a substituted heterogeneous group, a carbocyclic group, a substituted carbocyclic group, and heterocyclic group, a substituted heterocyclic group, an aromatic group, a substituted aromatic group, a heteroaromatic group, and a substituted heteroaromatic group;
each R⁹ is independently selected from the group consisting of a monovalent hydrocarbon group, a substituted monovalent hydrocarbon group, a heterogeneous group, a substituted heterogeneous group, a carbocyclic group, a substituted carbocyclic group, and heterocyclic group, a substituted heterocyclic group, an aromatic group, a substituted aromatic group, a heteroaromatic group, and a substituted heteroaromatic group;
p is an integer with a value of 0 to 6, q is an integer with a value of 0 to 5, with the proviso that (p+q)=1 to 5, and
bonds a, b, and c are each independently selected from the group consisting of a single bond, a cis double bond, and a trans double bond.

28. The method of claim 27, wherein W is selected from the group consisting of an oxygen atom and —(CH₂)ₘ—.

29. The method of claim 27, wherein X is OR⁸.

30. The method of claim 27, wherein Y is selected from the group consisting of a bond, an oxygen atom, and NHR⁸.

31. The method of claim 27, wherein Z is selected from the group consisting of aromatic, heteroaromatic, substituted aromatic, and substituted heteroaromatic groups.

32. The method of claim 27, wherein R¹ is selected from the group consisting of CO₂H, C(O)NHOH, CO₂R⁷, C(O)NHS(O)₂R⁷, and tetrazole.

33. The method of claim 27, wherein each R⁵ is independently selected from the group consisting of H and CH₃.

34. The method of claim 27, wherein each R⁶ is independently selected from the group consisting of H, CH₃, C₂H₅, and OR⁸.

35. The method of claim 27, wherein R⁷ is selected from the group consisting of methyl, ethyl, and isopropyl groups.

36. The method of claim 27, wherein each R⁸ is a hydrogen atom.

37. The method of claim 27, wherein p is an integer with a value 1 to 5,

38. The method of claim 27, wherein bond a is selected from the group consisting of a single bond and a cis double bond.

39. The method of claim 27, wherein bond b is selected from the group consisting of a single bond and a trans double bond.

40. The method of claim 27, wherein Y is a bond, p is 0, and q is 2 or 3.

41. The method of claim 27, wherein Y is a bond, q is 0, and component A) has the structure: 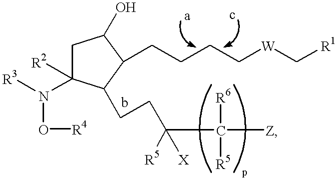
wherein R¹, W, R², R³, R⁴, R⁵, X, R⁶, Z, p, bonds a, b, and c are as described above.

42. The method of claim 27, wherein the composition is administered by a route selected from the group consisting of systemic and topical routes.

43. The method of claim 42, wherein the composition is a topical composition in a form selected from the group consisting of solutions, oils, creams, ointments, gels, lotions, shampoos, leave-on and rinse-out hair conditioners, milks, cleansers, moisturizers, sprays, and skin patches.

44. The method of claim 43, wherein the composition is a topical composition further comprisinga topical carrier comprising an ingredient selected from the group consisting of q) emollients, r) propellants, s) solvents, t) humectants, u) thickeners, v) powders, w) fragrances, water, alcohols, aloe vera gel, allantoin, glycerin, vitamin A and E oils, mineral oil, propylene glycol, polypropylene glycol-2 myristyl propionate, dimethyl isosorbide, and combinations thereof.

45. The method of claim 43, wherein the composition further comprises C) an activity enhancer selected from the group consisting of i) a hair growth stimulant, ii) a penetration enhancer, and combinations thereof.

46. The method of claim 45, wherein component i) is selected from the group vasodilator, an antiandrogen, a cyclosporin, a cyclosporin analog, an antimicrobial, an anti-inflammatory, a thyroid hormone, a thyroid hormone derivative, and a thyroid hormone analog, a non-selective prostaglandin agonist, a non-selective prostaglandin antagonist, a retinoid, a triterpene, and combinations thereof.

47. The method of claim 43, wherein component ii) is selected from the group consisting of 2-methyl propan-2-ol, propan-2-ol, ethyl-2-hydroxypropanoate, hexan-2,5-diol, polyoxyethylene(2) ethyl ether, di(2-hydroxypropyl) ether, pentan-2,4-diol, acetone, polyoxyethylene(2) methyl ether, 2-hydroxypropionic acid, 2-hydroxyoctanoic acid, propan-1-ol, 1,4-dioxane, tetrahydrofuran, butan-1,4-diol, propylene glycol dipelargonate, polyoxypropylene 15 stearyl ether, octyl alcohol, polyoxyethylene ester of oleyl alcohol, oleyl alcohol, lauryl alcohol, dioctyl adipate, dicapryl adipate, di-isopropyl adipate, di-isopropyl sebacate, dibutyl sebacate, diethyl sebacate, dimethyl sebacate, dioctyl sebacate, dibutyl suberate, dioctyl azelate, dibenzyl sebacate, dibutyl phthalate, dibutyl azelate, ethyl myristate, dimethyl azelate, butyl myristate, dibutyl succinate, didecyl phthalate, decyl oleate, ethyl caproate, ethyl salicylate, isopropyl palmitate, ethyl laurate, 2-ethyl-hexyl pelargonate, isopropyl isostearate, butyl laurate, benzyl benzoate, butyl benzoate, hexyl laurate, ethyl caprate, ethyl caprylate, butyl stearate, benzyl salicylate, 2-hydroxypropanoic acid, 2-hydroxyoctanoic acid, dimethyl sulphoxide, N,N-dimethyl acetamide, N,N-dimethyl formamide, 2-pyrrolidone, 1-methyl-2-pyrrolidone, 5-methyl-2-pyrrolidone, 1,5-dimethyl-2-pyrrolidone, 1-ethyl-2-pyrrolidone, phosphine oxides, sugar esters, tetrahydrofurfuryl alcohol, urea, diethyl-m-toluamide, 1-dodecylazacyloheptan-2-one, and combinations thereof.

48. The method of claim 43, wherein the topical composition locally administered on the skin once per day.

49. The method of claim 48, wherein the topical composition is administered once per day for 6 to 12 weeks.

50. A mascara composition comprising:
A) an active ingredient selected from the group consisting of oximyl- and hydroxylamino-prostaglandins having the functionality 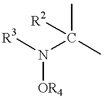
wherein C is a carbon atom bonded within a cyclopentyl ring and wherein the active ingredient selectively activates FP receptors and does not activate any other receptors that negate effects caused by activating the FP receptors, and wherein
R² is hydrogen, and R³ is selected from the group consisting of hydrogen and a lower monovalent hydrocarbon group, with the proviso that alternatively, R² and R³ may form a covalent bond, and
R⁴ is selected from the group consisting of a hydrogen atom, a monovalent hydrocarbon group, a heterogeneous group, a carbocyclic group, heterocyclic group, an aromatic group, a heteroaromatic group, a substituted monovalent hydrocarbon group, a substituted heterogeneous group, a substituted carbocyclic group, a substituted heterocyclic group, a substituted aromatic group, and a substituted heteroaromatic group,
dd) a water-insoluble material,
ee) a water-soluble, film-forming polymer,
ff) a wax;
o) a surfactant;
gg) pigment; and
s) a solvent.

51. A method for darkening and thickening hair, wherein the method comprises applying to growing hair and skin a composition comprising:
A) an active ingredient selected from the group consisting of oximyl- and hydroxylamino-prostaglandins having the functionality 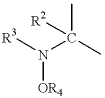
wherein in each functionality C is a carbon atom bonded within a cyclopentyl ring and wherein the active ingredient selectively activates FP receptors and does not activate any other receptors that negate effects caused by activating the FP receptors, and wherein
R² is hydrogen, and R³ is selected from the group consisting of hydrogen and a lower monovalent hydrocarbon group, with the proviso that alternatively, R² and R³ may form a covalent bond, and
R⁴ is selected from the group consisting of a hydrogen atom, a monovalent hydrocarbon group, a heterogeneous group, a carbocyclic group, heterocyclic group, an aromatic group, a heteroaromatic group, a substituted monovalent hydrocarbon group, a substituted heterogeneous group, a substituted carbocyclic group, a substituted heterocyclic group, a substituted aromatic group, and a substituted heteroaromatic group; and
B) a carrier.
